(19) United States
(12) Patent Application Publication
(10) Pub. No.: US 2022/0406401 A1
(43) Pub. Date: Dec. 22, 2022

(54) COMBINATION OF EXISTING DRUGS TO REPAIR THE ACTION POTENTIALS OF CELLS

(71) Applicant: Simula Innovation, Lysaker (NO)

(72) Inventors: Karoline Horgmo Jaeger, Oslo (NO); Aslak Tveito, Lysaker (NO)

(73) Assignee: Simula Innovation, Lysaker (NO)

(21) Appl. No.: 17/842,400

(22) Filed: Jun. 16, 2022

Related U.S. Application Data
(60) Provisional application No. 63/212,006, filed on Jun. 17, 2021.

(51) Int. Cl. G16B 5/00; A61K 45/06

(52) U.S. Cl. CPC G16B 5/00; A61K 45/06

(57) ABSTRACT

A process is provided for using a model to identify potential combinations of drugs to repair an action potential. A model is created for the normal state of an action potential along with a model for an abnormal state that reflects the effect of a disease or mutation on the action potential. Using a known action potential, a treated state can be generated that represents the abnormal state modified by one or more drug effects. By optimizing a formulation for a drug combination to minimize the differences between the treated state model and the normal state model, a combination of drug therapies that can potentially repair an action potential can be identified.

### CROSS-REFERENCE TO RELATED APPLICATIONS

This application claims the benefit and priority of U.S. Provisional Application No. 63/212,006, filed on Jun. 17, 2021, entitled “COMBINATION OF EXISTING DRUGS TO REPAIR THE ACTION POTENTIAL OF CELLS,” which is incorporated by reference herein in its entirety for all purposes.

### BACKGROUND

Poly-pharmacology (using multiple drugs to treat disease) has been proposed for improving cardiac anti-arrhythmic therapy for at least two decades. While disease and drug mechanisms are often well-understood, developing multi-compound therapies remains costly and time consuming. Screening new drug combinations for efficacy can require extensive testing, in part because effective drug combinations may be counterintuitive. Consequently, countless tests are often run on unsuccessful drug combinations in order to find a promising therapy. While many individual compound effects and disease pathogeneses are well-understood, this understanding has not led to rapid identification of effective drug combinations. Accordingly, it is desirable to develop a computer model to uncover therapeutic drug combinations without the need for trial and error of testing in a laboratory or clinical setting.

### BRIEF SUMMARY

Techniques are provided for determining viable (e.g., optimal) combinations of existing drugs to repair action potentials (APs). A model can be used to evaluate different compound combinations to determine if the combinations are potential multi-compound therapies for AP diseases, including heart conditions (e.g., cardiac arrhythmia) or neurological conditions (e.g., epilepsy). The concentrations of each compound can be varied to determine an optimum dosage.

In one embodiment, a compound's effect on the current density of a defined ion channels across a cell membrane can be determined. A normal action potential corresponding to a normal state of a cell and an abnormal action potential corresponding to an abnormal state of a cell can be identified. A model can be determined that outputs the action potential for an input of current densities of a set of ion channels across the cell membrane. The model can be used to determine a first combination of two or more of the set of compounds. For each compound in this first combination, the compound can be applied to the abnormal state of the cell to determine the modified current densities for specified ion channels for a given concentration of the compound. The modified current densities can be used to determine a treated action potential. A difference score can be determined by comparing the treated action potential and the normal action potential. The concentration of each compound in the first combination can be varied to determine an optimum difference score. Whether the first combination is a treatment for the disease can be determined by comparing the optimum difference score to a threshold value.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1A shows a graph of the AP transient biomarkers utilized in the cost function;

FIG. 1B shows Ca²⁺ transient biomarkers utilized in the cost function.

FIG. 2A shows the steady state I_{Kurd} in the control case and for three different doses of DPO-1 modeled using a Markov model and a EC₅₀ model and FIG. 2B shows the steady state I_{Kurd} in the control case and for three different doses of DPO-2 modeled using a Markov model and a EC₅₀ model.

FIG. 3A shows a graph of the effect on the I_{Kr} current using varying doses of HCQ.

FIG. 3B shows a graph of the effect using various doses of AZM and FIG. 3C shows the same effect using varying doses of the combination of HCQ and AZM.

FIG. 4A shows the geometry of ventricular myocytes used in EMI models in the adult CM and hiPSC-CM cases. FIG. 4B shows an illustration of the full EMI model domain, consisting of a strand of 100 connected myocytes and a surrounding extracellular space.

FIG. 5 is a table with characteristics of selected drugs in the form of EC₅₀ values, Hill coefficients (H), and maximum effects (E) for I_{Kr}, I_{CaL}, I_{Na}, I_{NaL}, and I_{f} currents.

FIGS. 6A-6I show simulated action potentials, Ca²⁺ transients and I_{Kr} currents generated for wild type and SQT1 hiPSC-CMs, rabbit ventricular cardiomyocytes (CMs) and adult human ventricular CMs.

FIGS. 7A-7C shows the optimal cost function values obtained using computational procedure to identify combinations of two drugs for repairing the SQT1 mutation in hiPSC-CMs, rabbit ventricular CMs and adult human ventricular CMs.

FIGS. 8A-8F show AP and Ca²⁺ transient for hiPSC-CMs, rabbit ventricular CMs and adult human ventricular CMs in the wild type case, in the SQT1 case, and in the SQT1 case with the optimal combination of two drugs.

FIG. 9 shows AP and Ca²⁺ transient for adult human ventricular myocytes in the wild type case, in the SQT1 case, and in the SQT1 case with the optimal dose of each of the drugs applied.

FIG. 10 is a table showing optimal doses of either a single drug or combinations of two drugs.

FIG. 11A shows optimal cost function values obtained when the computational procedure is applied to combinations of an increasing number of drugs with the goal of repairing the SQT1 mutation in hiPSC-CMs, FIG. 11B shows the cost function values for rabbit ventricular CMs and FIG. 11C shows the cost function values for adult human ventricular CMs.

FIGS. 12A-12F show AP and Ca²⁺ transient waveforms for hiPSC-CMs, rabbit ventricular CMs and adult human ventricular CMs in the wild type case, the SQT1 case, and the SQT1 case with the optimal combination of five drugs with the restriction D≤min(EC₅₀)/2 applied.

FIG. 13A is a table with cost function values and biomarkers for the SQT1 human ventricular CM model based on an optimal combination of five drugs with the restriction D≤min(EC₅₀)/2 applied and FIG. 13B shows optimal doses of a combination of five drugs with the same restriction.

FIG. 14 shows AP and Ca²⁺ transient for hiPSC-CMs in the wild type case, in the SQT1 case, and in the SQT1 case following application of an optimal dose of each of the drugs listed in FIG. 5 applied.

FIG. 15 shows AP and Ca²⁺ transient for rabbit ventricular CMs in the wild type case, in the SQT1 case, and in the SQT1 case with the optimal dose of each of the drugs of FIG. 5 applied.

FIG. 16 shows a flowchart illustrating a method for identifying an optimal drug combination for repairing an AP according to embodiments of the present disclosure.

FIG. 17 shows a block diagram of a computer system according to embodiments of the present disclosure.

### DETAILED DESCRIPTION

An action potential (AP) is the rapid rise and slow decay of voltage across a cellular membrane. The change in charge propagates along the cellular membrane and is a trigger for various physiological processes including cell communication and muscle contraction. The charge in an AP is generated by an ion differential across the cellular membrane. The sudden change in ion concentration that causes AP's characteristic electrical activity occurs when proteins (called ion channels) allow ions to flow across the cellular membrane. In mammals, APs occur in a variety of cell types including nerve cells, endocrine cells, and muscle cells, e.g., heart cells.

Neurons are an electrically excitable nerve cell that can be categorized as motor neurons, sensory neurons, or interneurons. Motor neurons stimulate muscle cells to contract while sensory neurons respond to stimuli (e.g., touch, smell, light, etc.) Interneurons allow communication from one neuron other neurons and the central nervous system (e.g., the brain and spinal cord) is primarily composed of interneurons. Diseases that alter neuron APs include epilepsy, episodic ataxia, familial hemiplegic migraine, Lambert-Eaton myasthenic syndrome, Alzheimer's disease, Parkinson's disease, schizophrenia, or hyperekplexia.

Cardiac cells include two types of electrically excitable cells: cardiomyocytes and cardiac pacemaker cells. Cardiomyocytes are the muscle cells in the heart that contract to circulate blood through the body. A cardiomyocyte contracts in response to an AP that propagates through the cardiac tissue. The cardiac AP is initiated by the cardiac pacemaker cells which is a specialized cell that regularly initiates an AP so that the cardiomyocytes contract in unison. Diseases that alter cardiac cell APs include Brugada syndrome, long QT-syndrome, short QT-syndrome, atrial standstill, or sick sinus syndrome. The present disclosure includes a systematic strategy for identification of new drugs or combinations of drugs, based on the selection of existing drugs. The new drugs or combination of drugs can be used to treat diseases that alter APs including epilepsy, episodic ataxia, familial hemiplegic migraine, Lambert-Eaton myasthenic syndrome, Alzheimer's disease, Parkinson's disease, schizophrenia, hyperekplexia Brugada syndrome, long QT-syndrome, short QT-syndrome, drug induced long QT, atrial fibrillation, atrial standstill, or sick sinus syndrome. Embodiments can use models of the AP and models of how drugs influence the AP's underlying ion currents.

For a group of compounds with known mechanism of action and a disease with a known pathogenesis, a model can be developed to test the combined effect of a set of compounds on an abnormal state. An abnormal state can be defined as a set of differences in the action potential between a diseased individual and someone without the condition. The differences between the two states can be identified by comparing an abnormal state to a normal state. An abnormal state can result from a genetic mutation or from lifestyle and environmental factors. A normal state can be represented by an action potential of a healthy subject with the wild type sequence (allele) of a gene.

Additionally, a combination's impact on an abnormal state can be determined by analyzing each compound's individual effect. A model can be developed to calculate a treated state that represents the combined effect of two or more drugs on a disease. This treated state can be compared to the normal state, and if the normal state and the treated state are sufficiently similar, the multi-compound therapy may be an effective treatment for the disease.

Also, the individual compound concentrations can be varied to determine an optimum concentrations for the multi-compound therapy's treatment dosage. If there is no two compound combination that effectively treats the disease, increasingly larger compound combinations (e.g., three or more compounds) can be modeled until an effective treatment is identified. In this way, a computer model can leverage compound and disease knowledge to identify potential multi-compound therapies. More specifically, embodiments can compute optimal, combined, drug concentrations such that the waveform of the transmembrane potential and the cytosolic calcium concentration of the mutant CMs can become as similar as possible to their wild type counterparts after the drug has been applied.

### I. Modeling the Action Potential

Mutations are known to cause perturbations in the essential functional features of integral membrane proteins, including ion channels. Even restricted or point mutations can result in substantially changed properties of ion currents. The additive effect of these alterations for a specific ion channel can result in significantly changed properties of the action potential (AP). Both AP shortening and AP prolongation can result from various mutations, and the consequences can be life-threatening.

A model for an AP can be created for a mutation where there are known changes to the ion channels involved in the AP. The AP can be modeled by simulating current and ion flow across a cellular membrane. The current flow can be determined through modeling the changes in ion concentrations across the cellular membrane. Multiple types of ion channels can be implicated in the flow of a particular ion and the effect of each type of ion channel can be determined independently as part of the action potential model.

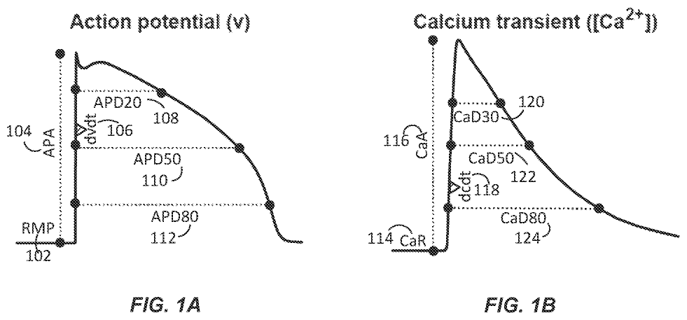

FIG. 1A shows a graph of current changes during a simulated cardiomyocyte (CM) AP according to embodiments of the present disclosure. The resting membrane potential (RMP) 102 is the cellular membrane voltage of an undisturbed cell. Once an AP has been initiated, the cellular membrane depolarizes from the RMP 102 until reaching the action potential amplitude (APA 104). The depolarization rate is reflected in the maximal upstroke velocity of the action potential (dvdt 106). The depolarized cellular membrane repolarizes until the membrane returns to the RMP 102. The action potential duration can be divided by how long it takes for the cellular membrane to repolarize with intervals shown for the action potential duration at 20% repolarization (APD20 108), 50% repolarization (APD50 110), and 80% repolarization (APD80 112).

FIG. 1B shows the simulated change in calcium ion concentration during a CM AP. The resting cystolic Ca²⁺ concentration (CaR) 114 is the calcium concentration within an undisturbed cell. Once an AP has been initiated, depolarization of the cellular membrane allows calcium to flow into the cell through ion channels until the calcium concentration reaches a maximum concentration reflected in the cytosolic Ca²⁺ ion transient amplitude (CaA 116). The calcium concentration changes according to the maximal ion transient upstroke velocity of the cytosolic Ca²⁺ concentration (dcdt 118). The calcium ion concentration is reduced until the concentration returns to CaR 114. The action potential duration can be divided by how long it takes for the calcium ion concentration to return to CaR 114 with intervals shown for the action potential duration at a 30% reduction in calcium amplitude (CaD20 120), 50% reduction in calcium amplitude (CaD50 122), and 80% reduction in calcium amplitude (CaD80 124). In other embodiments, the change in concentration of other ions, including potassium and sodium can be modeled.

A. AP Model States

Identifying optimal drug combinations can involve separate AP model types, e.g., three types: a model for a normal state, an abnormal state, and a treated state. The models can simulate the ion concentration, current, and voltage changes that occur during AP. The normal state and treated state can be compared to determine the differences between the model's ion concentration, current, and voltage changes. A combination of drugs (compounds) that changes an abnormal state to a treated state that is within an acceptable threshold of the normal state can be a potential treatment for the condition causing the abnormal state.

1. Normal State

The normal state model can replicate a healthy action potential with no disease or mutation and without any drug effects. Whether a particular model is a normal state depends on context and a single model can be a normal state or an abnormal state under different circumstances. For instance, one model may be a normal state model for an elderly man when attempting to develop a treatment for a geriatric disease, but that same model may be an abnormal state when attempting to correct the effect of aging on an AP. A normal action potential can be identified using in silico, in vitro (e.g., subcellular patch-clamp), or in vivo methods. In vivo refers to a process studied in a living organism, in vitro is the analysis of cells outside of their normal biological context (e.g., in a test tube), and in silico refers to a computer simulation of a biological process. Subcellular patch clamp is a laboratory technique in electrophysiology that allows the study of ionic currents in an isolated living cell. Some methods, such as genetically encoded calcium indicators (GECI, e.g., GCaMP) allow for the optical measurement of calcium ions in cells and GECIs can be used to determine a normal state of the action potential in vitro and in vivo.

2. Abnormal State

The abnormal state model replicates an action potential that differs from the normal model. The abnormal state could be caused by a mutation, disease, age, sex, drug effect, or any other condition that alters the AP. As described above in I.A.1, a single model could be a normal state and an abnormal state depending on context.

3. Treated State

The treated state model can be the abnormal state model modified by the modeled effect of one or more compounds on the AP. The effect of a number of drugs can be applied to the abnormal state model so that the treated state model is within an acceptable threshold of the normal state. A combination of drugs that creates a treated state that is similar to the healthy state can be a possible disease treatment.

B. Model of an AP

The various state models can be developed using model of an AP. Models of the action potential can be written in the form:

$$dv / dt = - {\sum }_{i} {I}_{i} \qquad ( 1 )$$

where ν is the membrane potential (in mV), t denotes time (in ms) and Iᵢ denotes membrane currents (in A/F). The membrane currents can include one or more of a fast sodium current (I_{Na}), a late sodium current (I_{NaL}), a transient outward potassium current (Iₜₒ), a rapidly activating potassium current (I_{Kr}), a slowly activating potassium current, an inward rectifier potassium current (I_{K1}), a hyperpolarization activated funny current (I_{f}), an L-type Ca²⁺ current (I_{CaL}), a background calcium current (I_{bCa}), a background chlorine current (I_{bCl}), a sodium-calcium exchanger current (I_{NaCa}), a sarcolemmal Ca²⁺ pump current (I_{pCa}), a sodium-potassium pump current (I_{NaK}) an applied stimulus current (Iₛₜᵢₘ), etc. Individual ion channel currents can be written on the form:

$${I}_{i} = {ρ}_{i} {J}_{i} , \qquad ( 2 )$$

$$where :$$

$${ρ}_{i} = \frac{{N}_{i}}{A {C}_{m}} \qquad ( 3 )$$

$$and :$$

$${J}_{i} = {g}_{0}^{i} {o}_{i} ( v - {E}_{i} ) . \qquad ( 4 )$$

Here, A can be the area of membrane surface membrane (in μm²), Cₘ can be the specific capacitance of the cell membrane (in pF/μm²), Nᵢ can be the number of channels of type i, g₀ⁱ is the conductance of a single open channel (in nS), oᵢ can be the unitless open probability of the channel, and Eᵢ can be the electrochemical equilibrium potential of the channel (in mV).

Splitting Iᵢ into ρᵢ and Jᵢ is convenient because it allows the effect of mutation and maturation/translation to be separated. It can be assumed that only ρᵢ is changed during maturation or by translation from animal cells to human cells. Likewise, only Jᵢ can be changed by the mutation. This can hold the other way around; ρᵢ can be independent of the mutation and Jᵢ can be independent of the maturation/translation.

Various embodiments can be based on various assumptions. The wild type (WT) and mutant (M) action potentials are well characterized by models. A family of K existing drugs have been identified and characterized in terms of how each of these compounds affects the currents in the AP model. Embodiments can apply simple IC₅₀/EC₅₀ models, or more comprensive Markov type models, to represent the effect of the compounds. The simple models of the action of each compound are multiplicative in the sense that the effect of several compounds can be multiplied in order to model the combined effect on a specific ion current. Using a model based on one or more of these assumptions, a therapeutic combination of the K different compounds can be identified.

C. EMI Model

The AP model described above provides a model for an action potential across a single membrane for a stand-alone cell. In some implementations, it is desirable to verify that the treatment identified using the AP model corrects a disease's clinical presentation. For example, cardiac diseases can be diagnosed through the measurement of an irregular electrocardiogram (ECG). While a multi-compound therapy can be identified using the AP model described above, a multi-cell model can be used to verify the multi-compound treatment's effectiveness. The impact of a corrected AP can be modeled in silico with an that approach represents the extracellular space (E), the cell membrane (M) and the intracellular space (I), see, e.g., [38, 39, 40]. The EMI is discussed further in section IV.A-IV.C.

### II. Compound Effects

A treated state model can involve a model for the effects of individual drugs or combinations of drugs on ion channels. The effect of a drug combination can be modeled by multiplying the effect of each constituent drug in the combination. The model presented herein allows the identification of optimal drug combinations in human cells using data from stem cell and animal models.

A. Single Drug Effects

For K different drugs, an optimal combination of these drugs can be found in order to develop a treated state and ‘repair’ the effect of a mutation or a condition. Finding an optimal drug combination can be achieved using a model of how each drug affects the properties of a mutant ion channel. The analysis can be based a simple model of drug effects (IC₅₀/EC₅₀) or more detailed Markov models. IC₅₀ is the concentration of a substance necessary to inhibit a biological process by 50% while EC₅₀ is the concentration needed to cause a response that is 50% of the maximum possible effect. IC₅₀ is measured for inhibitory substances while EC₅₀ is for excitatory substances. An excitatory substance can be a substance, such as an excitatory neurotransmitter, that increases the probability of an action potential. An inhibitory substance can decrease the probability of an action potential. Inhibitory substances can include inhibitory neurotransmitters. However, the same procedure is applicable if data is available to allow representations of drug effects using other models including Markov models.

A reasonable assumption is that both ion channel blockers (antagonists) and openers (agonists) can be encountered and therefore both cases should be addressed. To this end, the effect of a drug on a current I can be written in the form:

$$I ( D ) = ( 1 + \frac{{( ε D )}^{H}}{{( ε D )}^{H} + 1} E ) I ( 0 ) . \qquad ( 5 )$$

Here, D denotes the concentration of the drug, E is the maximum effect of the drug, H is the Hill coefficient, and EC₅₀=1/E is the concentration that gives half maximum effect of the drug. A Hill coefficient quantifies the degree of interaction between ligand binding sites, and the coefficient can quantify impact of a ligand binding to a receptor on the likelihood that other ligands will bind to that receptor. The relative change of the current due to the drug is given by:

$$η ( D ) = \frac{I ( D ) - I ( 0 )}{I ( 0 )} = \frac{{( ε D )}^{H}}{{( ε D )}^{H} + 1} E . \qquad ( 6 )$$

Given that η(0)=0, η(1/E)=E/2 and η(∞)=E. The parameters ε, H and E can be estimated from data describing how the drug affects the properties of ion currents of the cell carrying a mutation. A blocker can be a drug that binds to a receptor and prevents a ligand from binding to the receptor. If the drug is a blocker, it is often convenient to use E=−1 and then (5) takes the usual form of the IC₅₀ model:

$$I ( D ) = \frac{1}{{( ε D )}^{H} + 1} I ( 0 ) . \qquad ( 7 )$$

where 1/ε=IC₅₀. Note than in estimating E, there can be an obvious lower bound (E=−1), but there can be no obvious upper limit and can be estimated through the data fitting procedure.

For K different drugs and for each drug k, where Eᵢ^{k}, εᵢ^{k} Hᵢ^{k}, have been determined for each current i that contribute to the AP, as discussed above. As a consequence, the model of the treated state for an AP under the influence of a specific drug k can be given by

$$\frac{dv}{dt} = - {\sum }_{i} {I}_{i} ( D ) = {\sum }_{i} ( 1 + \frac{{( {ε}_{i}^{k} {D}^{k} )}^{{H}_{i}^{k}}}{{( ε {D}^{k} )}^{{H}_{i}^{k}} + 1} {E}_{i}^{k} ) {I}_{i} ( 0 ) . \qquad ( 8 )$$

B. Drug Combination Effects

Many drugs can be combined while searching for an optimal multi-compound therapy, and by applying a combination of K drugs to the i-th current in the mutant model, it can be found that:

$${I}_{i} ( D ) = {\prod }_{k = 1}^{K} ( 1 + \frac{{( {ε}_{i}^{k} {D}^{k} )}^{{H}_{i}^{k}}}{{( {ε}_{i}^{k} {D}^{k} )}^{{H}_{i}^{k}} + 1} {E}_{i}^{k} ) {I}_{i} ( 0 ) . \qquad ( 9 )$$

In order to simplify this notation, the properties of the k-th drug can be denoted by Δₖ={Eᵢ^{k}, εᵢ^{k} Hᵢ^{k}} where i runs over all the transmembrane currents. Furthermore, if Δ denotes the combination of the K drugs given by {Δₖ}ₖ₌₁^{K}. The vector of doses is given by D={Dₖ}ₖ₌₁^{K}.

The AP model after the combination of drugs has been applied now takes the form:

$$\frac{{dv}^{Δ}}{dt} = - {\sum }_{i} {F}_{i} ( D ; Δ ) {I}_{i} ( 0 ) , \qquad ( 10 )$$

$$where :$$

$${F}_{i} ( D ; Δ ) = {\prod }_{k = 1}^{K} ( 1 + \frac{{( {ε}_{i}^{k} {D}^{k} )}^{{H}_{i}^{k}}}{{( {ε}_{i}^{k} {D}^{k} )}^{{H}_{i}^{k}} + 1} {E}_{i}^{k} ) . \qquad ( 11 )$$

C. The Channel Block/Agonist Model is Unchanged During Maturation or Species Translation

The properties {Eᵢ^{k}, εᵢ^{k} Hᵢ^{k}} of a drug k on a specific ion channel i can be assumed to be the same for animal and human cells. See [33]. The methods described in this disclosure could therefore be applied to find optimal drug compounds for adult human cells based on data from stem cell derived cells (e.g., hiPSC-CMs) or an animal (e.g., rabbit). Recall that the currents in the model are written on the form I=ρJ where the factor ρ changes during maturation, but can be unaltered by the mutations; and, vice versa. That is, the function J can be unchanged by maturation but is altered by the mutation. For a given ion current:

I^{IM,M}=ρ^{IM}J^{M} (12)

I^{A,M}=ρ^{A}J^{M}, (13)

where IM, A, and M can be for immature, adult and mutant, respectively. Recall that for an ion channel, J=g₀o(ν-E), and, under the influence of the drug, J=J(D)=F(D)g₀o(ν-E). Since J can be the same for IM and A, the effect of the drug can also be the same, and thus:

I^{IM,M}(D)=ρ^{IM}J^{M}(D), (14)

I^{A,M}(D)=ρ^{A}J^{M}(D). (15)

Therefore, if ε, H and E in the model (5) are estimated from measurements of hiPSC-CMs (e.g., using the computational inversion procedure of [32]), these values are also the correct values in the adult case. Exactly the same argument can be used to translate from rabbit data to E, H and E values for adult human CMs. The data can be translated because the effect of the drug on a specific ion channel can be the same regardless of whether it is expressed in hiPSC myocytes, rabbit myocytes or myocytes from adult humans.

D. Computational Maturation and Translation

As mentioned above some embodiments can employ a base AP model [32] that uses the same representation of a specific single channel, independent of the specie we are considering. Suppose a specific current for hiPSC-CMs are given by I^{IM}=ρ^{IM}J and the associated current for the adult CM is given by I^{A}=ρ^{A}J. Then we have I^{A} (1+λ)^{IM} where λ=(ρ^{A}−ρ^{IM})/ρ^{IM} is the maturation parameter. In exactly the same manner, we can define a translation parameter for mapping between a specific adult human current and the analogous current for an animal (rabbit in our case).

The specific model used below in section IV is an updated version of the base model [32]. We use the updated base model formulation described [12]. In that model, the I_{Kr} current is fitted to data from measurements of wild type and SQT1 I_{Kr} currents from [34]. Furthermore, an hiPSC-CM version of the model is fitted to data of wild type and SQT1 hiPSC-CMs from [28], and an adult human ventricular CM version of the model was validated using adult human ECG measurements from [35]. For the computations we also consider a rabbit version of the AP model. The rabbit parameterization is based on the rabbit models from [33, 36] and is fitted to published SQT1 and wild type APD90 values for rabbits from [37]. The parameters of the rabbit version of the model are specified in Table 1 in the Supplementary information section below, and the remaining parameter values of the base model are found in [12].

E. Comparison of IC₅₀-Based Modeling of Drug Effects to Markov Models of Drug Effects

More detailed models for drug effects can be used. Typically, these models are expressed in the form of Markov models (see, e.g., [24, 27]). An advantage of these more detailed models is that they are able to give a detailed representation of the effect of drugs, including, e.g., voltage and use-dependent drug effects. On the other hand, a disadvantage is that the Markov models typically introduce a large number of parameters that can be parameterized, that can be based on information from detailed measurements of the effect of the drug on the ion channels.

In order to begin to assess of the difference between a Markov model for drug effects and the more simplified IC₅₀-based modeling used in our study, we consider a Markov model for the I_{Kurd} current from [69]. Based on measurements of the effect of two drugs (DPO-1 and DPO-2) on this current, four different model parameterizations for the drugs were considered, see [69].

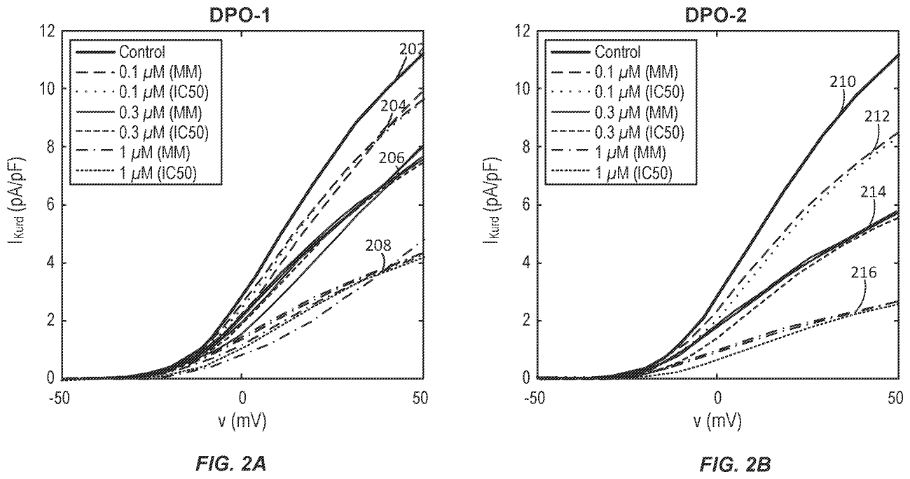

FIG. 2A shows a plot of the steady-state I_{Kurd} currents as a function of the membrane potential in the control 202 case and for three different doses of DPO-1 (0.1 μM 204, 0.3 μM 206, and 1 μM 208). The solid lines show the solution for each of the four Markov model parameterizations. In addition, the dotted lines show the currents computed using a simple IC₅₀-based scaling of the control current.

FIG. 2B shows a plot of the steady-state I_{Kurd} currents as a function of the membrane potential in the control 210 case and for three different doses of DPO-2 (0.1 μM 212, 0.3 μM 214, and 1 μM 216). We observe that the IC₅₀-based modeling of the drug effects is in relatively good agreement with the full Markov model of drug effects. Because of this and since detailed measurements of drug effects that can be used to set up realistic Markov model representations of drugs effects are not always available, we have chosen to use a simple IC₅₀-based modeling of drug effects in this study. However, various embodiments may use various models, such as the Markov models described above.

F. Effect of Separate Drugs Multiplicative?

A central assumption in the disclosed method is that if two drugs affect the same ion channel, their combined effect can be identified by multiplication of the respective effects, see (9) above. Using this assumption, effective drug combinations have been found. A recent paper [70] analyzes the effect of hydroxychloroquine (HCQ) and azithromycin (AZM) can be analyzed.

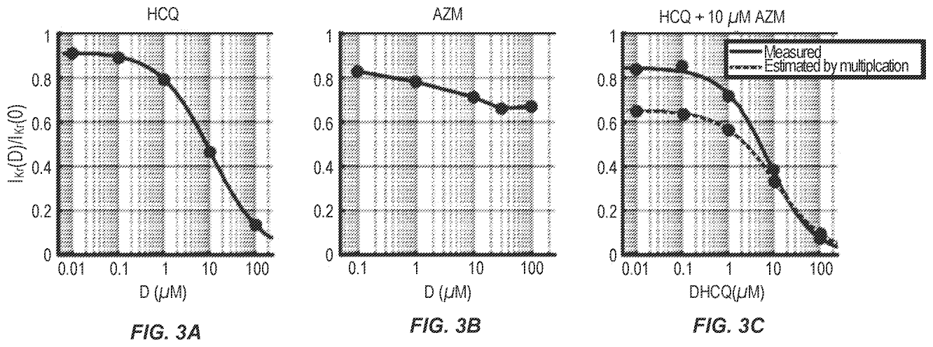

FIG. 3A shows the data from [70] on the effect on the I_{Kr} current by applying increasing doses of HCQ.

FIG. 3B shows the data from [70] on the effect on the I_{Kr} current by applying increasing doses of AZM.

FIG. 3C shows the data from [70] on the effect on the I_{Kr} current by applying increasing doses of a combination of HCQ and AZM. FIG. 3C also shows the estimated effect obtained by assuming that the combined effect of two drugs are multiplicative. The estimated results shown in FIG. 3C fit the measured results well, particularly at high doses.

### III. Identifying an Optimal Combination of Compounds

Developing a treatment using this model can involve identifying a set of optimal doses D={Dₖ}ₖ₌₁^{K} for a set of K different drugs with known properties Δₖ={Eᵢ^{k}, εᵢ^{k} Hᵢ^{k}} so that the treated state model of the drug-treated mutant cells with action potentials very closely approximate the transmembrane potential and calcium transient of the normal state model. To this end, an action potential model where the effect of the mutation can be represented such that an abnormal state model and normal state model are defined and can be easily compared is used. Furthermore, the optimal doses D can be estimated by minimizing a cost function measuring the difference between the model solutions.

A. Cost Function Definition

The following cost function can be utilized:

$$C ( D ) = {\sum }_{j} {w}_{j} \frac{\vert {R}_{j}^{M} ( D ) - {R}_{j}^{W} \vert}{\vert {R}_{j}^{W} \vert} , \qquad ( 16 )$$

where Rⱼ^{W} represent different biomarkers for the wild type AP model, Rⱼ^{M}(D) represent the corresponding biomarkers for the mutant model with the drug doses D applied, and wⱼ are weights for the different biomarkers. The cost function can represent a difference score between the treated action potential and the normal action potential.

B. Minimization Procedure

The problem of identifying the minimum of cost function (16) can grow in complexity as the number of drugs increases. Here, an approach that gradually increases the number of drugs is used and thus there can be a reasonably good initial guess for every minimization problem.

In the case of one drug, the optimum dose can be found by minimizing the cost function (16) with only one free parameter (the dose of the single drug). Suppose the optimal combination of n drugs is found (where n<K). Next, whether one of the remaining K-n drugs can improve the treated state's approximation of the normal state (wild type case) is considered. The problem is then to solve K-n minimization problems with n+1 parameters. The minimization is now started using a solution for n drugs as an initial guess, and for the one additional drug, the initial dose can be set to zero. A solution of this n+1 dimensional problem can be solved using the continuation method of [32, 12]. This is repeated for the K-n remaining drugs, and the solution can be stored as the optimal drug for the case of combining n+1 drugs. The process can be repeated until n=K. The optimized difference score (e.g., the minimized cost function) can be compared to a threshold to determine if the combination of compounds is an effective treatment for the condition or disease causing the abnormal state. The threshold can correspond to a minimum percentage similarity (or other measure of minimum similarity) to the normal action potential. The threshold can correspond to a minimum percentage similarity to the normal action potential if the threshold is a minimum percentage similarity or if the threshold is a weighted minimum percentage similarity multiplied by one or more weights. Further technical specifications of the applied minimization procedure are provided below in section III.C.

C. Technical Specifications of the Optimization Method

In order to find the drug doses that minimize the cost function (16), a continuation-based optimization method applied in [32, 83, 12] can be used. See, e.g., [12] for a detailed description of this method.

In the search for optimal doses of one or two drugs, doses can be restricted so that the maximal effect of the drug is at most a particular percentage (e.g., 95%) of the compound's maximal possible effect (E, see section II.A above). Optimization can involve continuation iterations (e.g., 20) with a specific number of combinations (e.g., 96, which may be randomly chosen) of doses in each iteration, and Nelder-Mead iterations (e.g., 15) can be run for each randomly chosen combination. This can be done for each application of the continuation algorithm in this procedure.

The number of considered drugs can gradually increase if no optimal combination of compounds (e.g., drugs) is found for a given number of combined compounds. The optimal combination of compounds can include one or more of disopyramide, ivabradine, veratridine, BAY K 8644, quinidine, amiodarione, propafenone, mexiletine, ajmaline, phenytoin, phenobarbital, carbamazepine, oxcarbazepine, gabapentin, pregabalin, lacosamide, vigabatrin, valproic acid, lamotrigine, topiramate, zonisamide, levetiraacetam, clonazepam, rufinamide, fulnarizine, dalfampridine, acetoazolamide, prednisolone, azathioprine, methotrexate, donepezil, rivastigmine, galantamine, memantine, levodopa, carbidopa, istradefylline, safinamide, pramipexole, rotigotine, ropinirole, amantadine, benztropine, trihexyphyenidyl, selegiline, rasagiline, entacapone, tolcapone, opicapone, choroprozamine, fluphenazine, haloperidol, perphenazine, thioridazine, thiothixene, trifluoperazine, aripiprazole, aripiprazole lauroxil, asenapine, brexpiprazole, cariprazine, clozapine, iloperidone, lumateperonee, lurasidone, olanzapine, samidorphan, paliperidone, paliperidone palmitate, quetiapine, risperidone, ziprasidone, diazepam, hydroxytryptophan, piracetam, sodium valproate, nifedipine, flecainide, etc.

### IV. Example Application—Short Qt Syndrome

The embodiments described herein can be used to compute an optimal multi-compound therapy for the Short QT syndrome type 1 (SQT1). For the SQT1 mutation, there are available datasets that describe the effect of various drugs on the mutated K⁺ channel. These data are the basis for the SQT1 abnormal states and the computational analysis presented below, which can identify optimal compounds so that the treated state of a cardiomyocyte resembles the normal state of a cardiomyocyte. Optimal multi-compound therapies are computed for hiPSC-CMs, rabbit ventricular cardiomyocytes (CMs) and adult human CMs with the SQT1 mutation. Since the ‘composition’ of ion channels that form the AP is different for the three types of myocyte abnormal states under consideration, so is the composition of the optimal multi-compound therapies used to generate the treated states.

SQT1 is a rare form of cardiac arrhythmia that results from mutations to the human Ether-a-go-go Related Gene (hERG) potassium channel. Functionally, these mutations permit the hERG channel to open earlier during each heart beat. Here a suite of CM computational models have been applied to determine whether multi-compound therapies may offer a more effective therapeutic strategy in SQT1. The analyses suggest that simultaneous induction of late sodium current and partial hERG blockade offers a promising strategy. While no activators of late sodium current have been clinically approved, several experimental compounds are available and may provide a basis for interrogating this strategy. Some embodiments presented here can be used to compute optimal drug combinations provided that the effect of each drug on relevant ion channels is known or can be determined.

A. Base Model Specifications

We use the base model formulation from [12] to represent the AP and calcium transient of normal state (wild type) and abnormal state (SQT1) cardiomyocytes. The full base model formulation and the specific hiPSC-CM and adult human ventricular CM parameterizations of the model are specified in [12]. A rabbit ventricular myocyte version of the model is also used. The parameters of the rabbit model are as specified for the adult human version of the model in [12], except for the parameter values specified in Table 1.

TABLE 1

| Parameter | Value (Rabbit) | | Value (Adult) | | Value (hiPSC-CM) | |
|---|---|---|---|---|---|---|
| g_{Na} | 3.78 | mS/μF | 5.04 | mS/μF | 1.38 | mS/μF |
| g_{Kr} | 0.048 | mS/μF | 0.025 | mS/μF | 0.126 | mS/μF |
| g_{K1} | 0.925 | mS/μF | 0.37 | mS/μF | 0.045 | mS/μF |
| Ī_{NaK} | 0.864 | μA/uF | 1.13 | μA/μF | 0.10 | μA/μF |
| g_{CaL} | 0.27 | nL/(μF ms) | 0.216 | nL/(μF ms) | 0.497 | nL/(μF ms) |
| Ī_{pCa} | 0.068 | μA/μF | 0.34 | μA/μF | 0.77 | μA/μF |
| gₜₒ | 0.189 | mS/μF | 0.27 | mS/μF | 0.084 | mS/μF |
| g_{Ks} | 0.06 | mS/μF | 0.035 | mS/μF | 0.037 | mS/μF |
| g_{bCl} | 0.0163 | mS/μF | 0.0102 | mS/μF | 0.0113 | mS/μF |
| Ī_{NaCa} | 17.64 | μA/μF | 4.9 | μA/μF | 13.5 | μA/μF |
| g_{bCa} | 0.000132 | mS/μF | 0.00039 | μA/μF | 0.000056 | μA/μF |

Table 1 shows parameter values of the rabbit, adult human, and hiPSC-CM versions of the base model; the remaining parameter values are the same as for the adult human model in [12].

For the hiPSC-CM, adult human and rabbit versions of the base model pacing frequencies are used(e.g., 0.2 Hz, 1 Hz and 2 Hz). In the EMI model simulations, we run an ODE simulation (e.g., 500 pacing cycles) to update the initial conditions for each parameter change before starting the EMI model simulation. In the inversion procedure, we run a simulation (e.g., 50 or 100 pacing cycles for each of 20 or 5 iterations of the continuation method), and update the initial conditions in each iteration (see [12]).

B. Technical Specifications of the EMI Model Simulations

An EMI model is useful for verifying that the multi-compound therapy identified through the AP model actually treats the disease in question. Specifically, SQT-1 presents as a shortened QT interval in an ECG. The EMI allows for a simulated ECG using treated state cells by simulating an action potential propagating through cardiac tissue composed of treated state cells. While a multi-compound therapy is identified by looking at the AP, the therapy's efficacy can be verified using an EMI model.

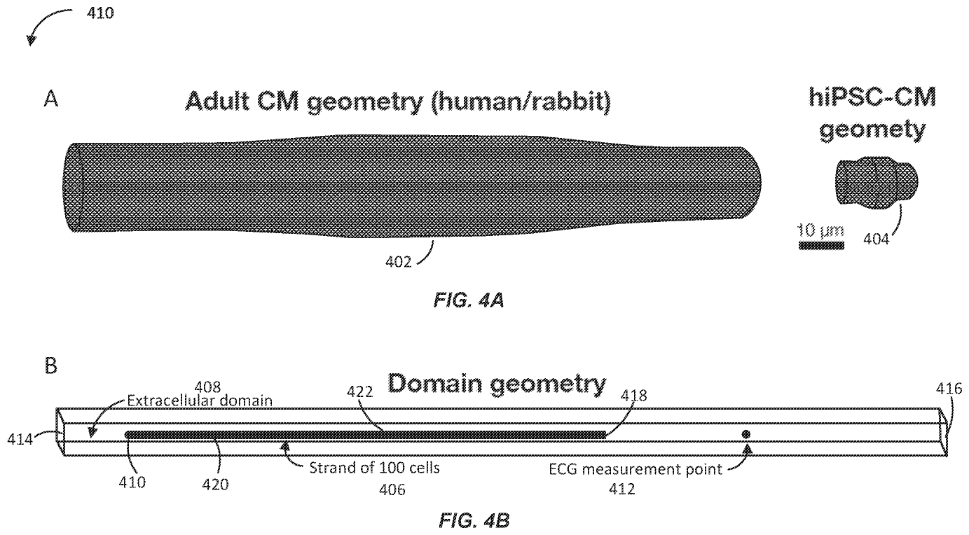

FIG. 4A shows the geometry of a single myocyte. In the adult case, each adult myocyte 402 is 150 μm long and has a radius varying from 8 μm to 11 μm. In the hiPSC-CM case, each hiPSC-CM myocyte 404 is 15 μm long and has a radius varying from 4 μm to 6 μm. In addition, we increase the gap junction resistance, R_{g}, in the hiPSC-CM case, representing a weaker gap junction coupling between hiPSC-CMs as compared to adult CMs (see, e.g., [84, 85]). More specifically, R_{g} is increased by a factor of 4, resulting in a default conduction velocity of approximately 5 cm/s, similar to what was observed in sheets of hiPSC-CMs in [86, 87, 88]. The parameters applied in the EMI model simulations (see, e.g., [43]) are specified in Table 2.

FIG. 4B shows the EMI domain geometry. We consider a strand of 100 connected myocytes 406 surrounded by an extracellular domain 408. We initiate a propagating AP through the myocytes by setting the intracellular potential of the first two (leftmost 410) myocytes to −10 mV. We apply a homogeneous Dirichlet boundary condition at the extracellular left boundary 414 and homogeneous Neumann boundary conditions on the remaining extracellular boundaries. The minimum distance from the extracellular boundary to the myocyte membrane in the y- and z-directions is 5 μm in the adult case and 10 μm in the hiPSC-CM case. The distance from the extracellular left boundary 414 to the first myocyte 410 is 0.5 cm and the distance from the extracellular right boundary 416 to the last myocyte 418 is 1.5 cm.

TABLE 2
Parameter values of the EMI
model simulations, see, e.g., [43].

| Parameter | Value | |
|---|---|---|
| Cₘ | 1 | μF/cm² |
| C_{g} | 0.5 | μF/cm² |
| σᵢ | 4 | mS/cm |
| σₑ | 20 | mS/cm |
| R_{g} | 0.0015 | kΩcm² |
| Δt | 0.005 | ms |
| Δt_{ODE} | 0.001 | ms |

| | |
|---|---|
| Mᵢₜ, Nᵢₜ | 1 |

The pseudo-ECG is measured in a point 412 1 cm to the right of the last myocyte (only for the adult human and rabbit cases). In order to set up a pseudo-ECG in these cases, we introduce transmural heterogeneity along the myocyte strand. More specifically, we let the first 25 myocytes define an endocardial region (extending from 410 to 420), the next 35 myocytes define a midmyocardial region (extending from 420 to 422) and the last 40 myocytes define an epicardial region (extending from 422 to 418). The default base model parameters define the parameters of the epicardial region (extending from 422 to 418). In the endocardial region (extending from 410 to 420), the I_{Ks} and Iₜₒ current densities are reduced to 31% and 1%, respectively, compared to the epicardial region. In the midmiocardial region (extending from 420 to 422), the currents are reduced to 11% and 85%, respectively [89].

The QT interval is computed as the time from the start of the QRS-complex of the computed pseudo-ECG to the end of the T-wave. More specifically, we extract the first and last time points of the simulation when the absolute value of the extracellular potential in the measurement point is above 1% of the maximum absolute value achieved at the measurement point.

The conduction velocity is computed as the distance between the center of myocyte number 70 and myocyte number 20 divided by the duration of the time interval between the membrane potential in the center of the two myocytes reaches a value above 0 mV.

C. Drug Characteristics

In this study, we attempt to identify optimal combinations of drugs for repairing the effect of the SQT1 mutation N588K, which alters the function of the potassium current I_{Kr}. Specifically, this mutation markedly increases the size of the I_{Kr} current, leading to a shortening of the AP. In order to ‘repair’ this effect, a number of I_{Kr} blockers are evaluated in an attempting to reduce the I_{Kr} current. In addition, two drugs that increase the I_{CaL} or I_{NaL} currents, are considered as alternative approaches for lengthening the action potential duration in the ventricular myocytes carrying this mutation.

### Computational Complexity

TABLE 3

| | hiPSC-CMs | | | Rabbit CMs | | | Adult human CMs | | |
|---|---|---|---|---|---|---|---|---|---|
| | N_{E} | N_{M} | N_{I} | N_{E} | N_{M} | N_{I} | N_{E} | N_{M} | N_{I} |
| Number of nodes | 9,535 | 7,016 | 7,754 | 13,032 | 6,616 | 7,198 | 13,032 | 6,616 | 7,198 |

| Number of state | 2*25 | | 2*25 | | 2*25 | |
|---|---|---|---|---|---|---|
| variables for M, N_{S} | | | | | | |
| Simulation time | 500 | | 250 | | 350 | |
| (ms) | | | | | | |
| | Δt_{PDE} | Δt_{ODE} | Δt_{PDE} | Δt_{ODE} | Δt_{PDE} | Δt_{ODE} |
| Time step (ms) | 0.005 | 0.001 | 0.005 | 0.001 | 0.005 | 0.001 |
| Number of time | N_{PDE} | N_{ODE} | N_{PDE} | N_{ODE} | N_{PDE} | N_{ODE} |
| points | 100,000 | 500,000 | 50,000 | 250,000 | 70,000 | 350,000 |

| | | | |
|---|---|---|---|
| Total number of | 4*8.9 · 10¹⁰ | 4*4.2 · 10¹⁰ | 4*5.9 · 10¹⁰ |
| computed values, | | | |
| (N_{E} + N_{I})N_{PDE} + | | | |
| N_{M}N_{S}N_{ODE} | | | |

Table 3 shows sizes of the computational problems associated with the EMI model simulations. The first row reports the number of finite element nodes in the extracellular (E), membrane (M) and intracellular (I) domains. The second row reports the number of state variables of the membrane model. The third row reports the simulation times used in the simulations of hiPSC-CMs, rabbit CMs and adult human CMs, respectively. The lower rows report the time step used for the ordinary differential equation (ODE) part of the simulation (for the state variables of the membrane) and the partial differential equation (PDE) part of the simulation, and the associated number of time steps for the full simulation time. Finally, the bottom row reports the total number of solution values computed during these simulations.

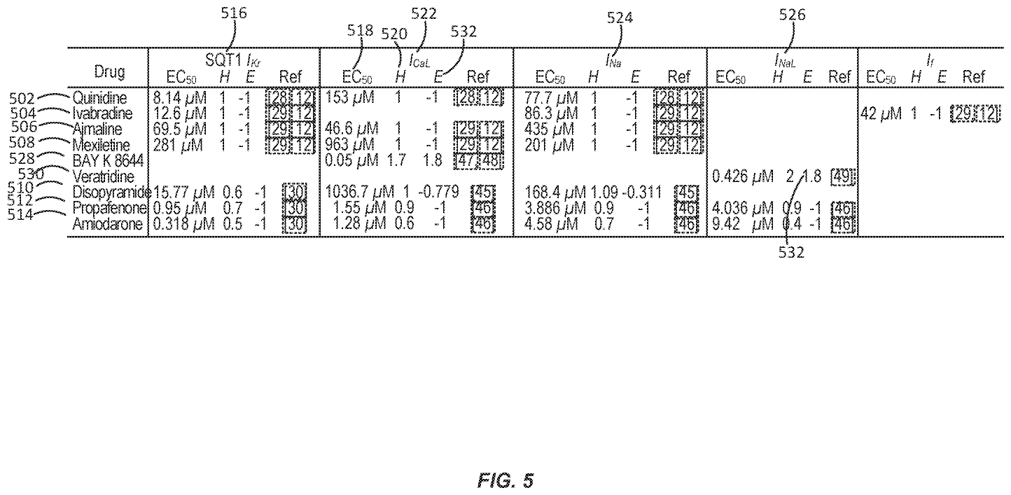

FIG. 5 lists the properties of the considered drugs. Here, the properties of the drugs quinidine 502, ivabradine 504, ajmaline 506 and mexiletine 508 are taken from Karoline Horgmo Jaeger et al., PLoS Computational Biology, 17(2): e1008089, 2021, where they were estimated based on measurements of SQT1 hiPSC-CMs from Ibrahim El-Battrawy et al. Journal of the American Heart Association, 7(7): e007394, 2018; and Zhihan Zhao et al., Clinical Pharmacology & Therapeutics, 106(3):642-651, 2019. Furthermore, the effect of the drugs disopyramide 510, propafenone 512 and amiodarone 514 on SQT1 I_{Kr} currents 516 are taken from MJ McPate et al., British Journal of Pharmacology, 155(6):957-966, 2008. The EC₅₀ values 518 are taken directly from the paper and the Hill coefficients 520 are estimated from fitting the model (5) to the dose-dependent block reported in FIGS. 4 and 5 of MJ McPate et al. Data describing the effect 532 of these drugs on I_{CaL} 522, I_{NaL} 524, and I_{NaL} 526 are taken from the comprehensive drug studies J Kramer et al., Scientific Reports, 3:2100, 2013; and W J Crumb et al., Journal of Pharmacological and Toxicological Methods, 81:251-262, 2016. Focused Issue on Safety Pharmacology., 46]. Finally, parameters describing the properties of the I_{CaL} and I_{NaL} agonists BAY K 8644 528 and veratridine 530 are relatively rough estimates based on data presented in Martin Bechem and Matthias Schramm, Journal of Molecular and Cellular Cardiology, 19:63-75, 1987; Gunter Thomas et al., Circulation Research, 56(1):87-96, 1985; and Wesley L McKeithan et al., Frontiers in Physiology, 8:766, 2017

D. Simulation Results

The simulations demonstrate that mathematical models disclosed herein can be used to find optimal multi-compound therapies for anti-arrhythmic treatments. It is shown, that theSQT1 mutation N588K abnormal state can be repaired to generate a treated state by applying the optimal combination of existing drugs.

1. Normal State and Abnormal State (SQT1 Mutation) in hiPSC-CMs, Rabbit CMs and Adult Human CMs

A normal state and abnormal state may need to be developed before attempting to identify compounds that can produce an acceptable treated state. The normal state (wild type and abnormal state (SQT1) were modeled in stem cells (hiPSC-CMs), rabbit cells, and adult human cells.

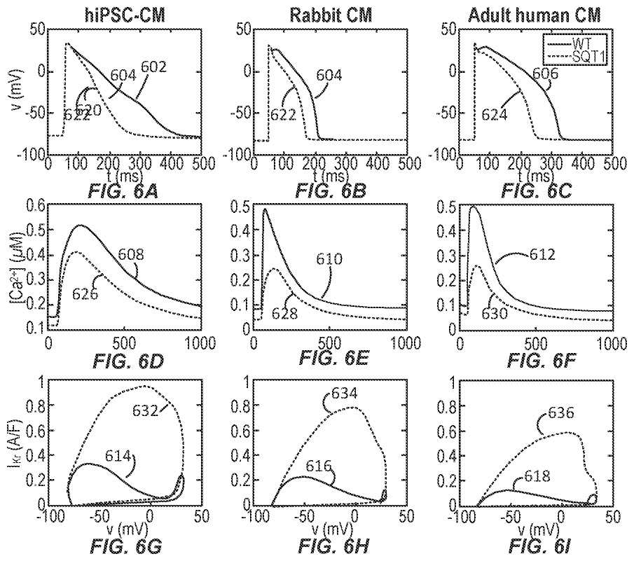

FIGS. 6A-6I show the AP FIGS. 6A-6C Ca²⁺ transient FIGS. 6D-6F and I_{Kr} currents FIGS. 6G-6I generated by the normal state (wild type 602-618) and abnormal state (SQT1 620-636) versions of the models for hiPSC-CMs FIGS. 6A, 6D, 6G, rabbit ventricular CMs FIGS. 6B, 6E, 6H and human adult CMs FIGS. 6C, 6F, 6I. The AP FIGS. 6A-6C is significantly shorter in the abnormal state (SQT1 620-624) than in the normal state (wild type 602-606) for all of the models.

The SQT1 mutation affects the function of the I_{Kr} current; a difference between the normal state (wild type) and abnormal state (SQT1) versions of the AP models is a difference in the formulation of the I_{Kr} current, see [12]. FIGS. 6G-6I compare the normal state (wild type 614-618) and abnormal state (SQT1 632-636) I_{Kr} currents by plotting these currents from the two action potential simulations as functions of the membrane potential. Note that the I_{Kr} current is significantly larger in the abnormal state (SQT1 632-636) than in the normal state (wild type 614-618). The voltage dependence is different in the abnormal state compared to the normal state.

The difference in voltage dependence indicates that drug effects implemented only in terms of altered maximum conductance, resulting from a pore block approach, for the I_{Kr} current FIGS. 6G-6I of the form (5) will likely not completely eliminate the effect of the SQT1 mutation on the I_{Kr} current FIGS. 6G-6I. Accordingly, instead of trying to repair the mutated I_{Kr} current directly, the model instead attempts to ‘repair’ the effect of the mutation on the full action potential by minimizing the cost function (16).

2. Treated State for Two Drugs

The computational procedure was first applied to search for a treated state by finding optimal combinations of two drugs for repairing the AP and Ca²⁺ transient of the abnormal state (SQT1 CMs).

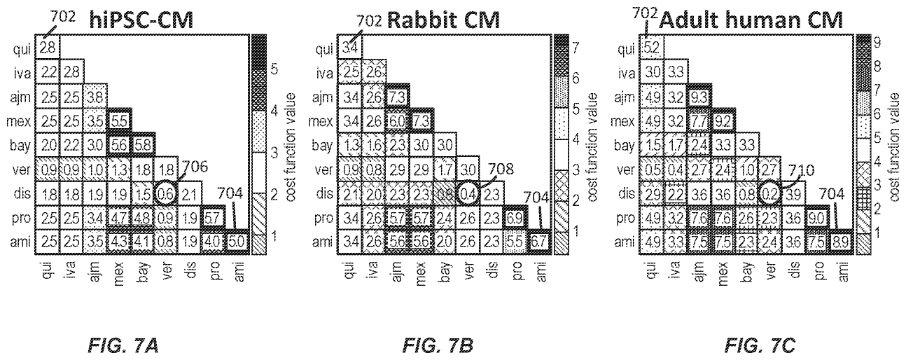

FIG. 7A-7C illustrates the findings presented in terms of the minimum cost function value (12) for the procedure applied to each possible combination of two drugs. In addition, the numbers along the diagonal from the upper left 702 to lower right 704 report the optimal cost function values found in searches for the optimal dose of each single drug. Note that some of the combinations of drugs appear to result in relatively low cost function values, and that the optimal combinations of two drugs appears to result in considerably lower cost function values than the optimal doses of any single drug. In particular, the combination of veratridine and disopyramide 704-710 in FIG. 7A-7C, appears to be the optimal combination of two drugs for both hiPSC-CMs FIG. 7A, rabbit ventricular CMs FIG. 7B and adult human ventricular CMs FIG. 7C. Neither of these drugs, individually, appear to be able to repair the effect of the mutations.

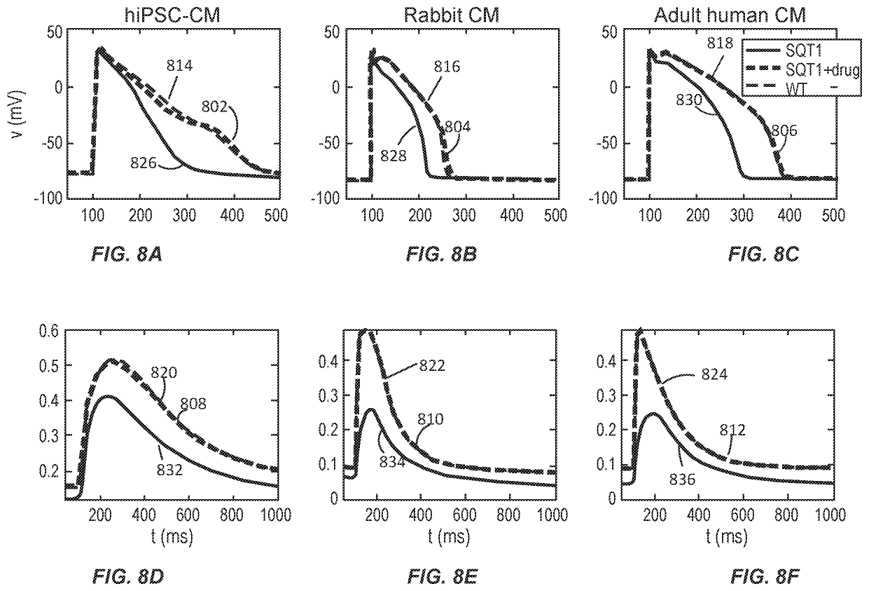

FIGS. 8A-8F shows the AP 8A-8C and Ca²⁺ 8D-8F transient of the treated state (SQT1 models under the influence of the optimal dose combination of these two drugs 802-812). FIGS. 8A, 8D show hiPSC-CM treated state 802, 807, FIGS. 8B, 8E show the rabbit ventricular CM treated state 804, 810 and FIGS. 8C, 8F show the adult human ventricular CM treated state 806, 812, and compare AP and Ca²⁺ transients for normal state (wild type 814-824), abnormal state (SQT1 826-836) and treated state (SQT1 with the drugs applied 802-812). The treated state with the optimal combination of two drugs 814-824 appears to repair the SQT1 AP and Ca²⁺ abnormal state 826-836 very well; that is the treated state SQT1 models with the optimal drug combination applied 802-812 seems to be very similar to the normal state wild type solutions 814-824.

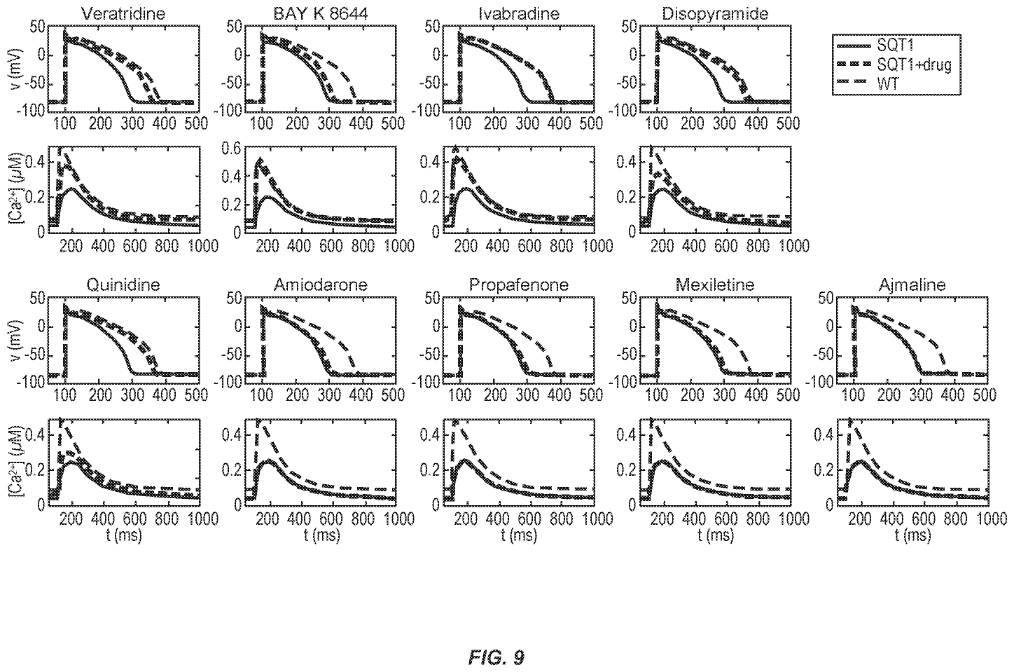

FIG. 9 shows similar plots for the optimal dose of each of the individual drugs in the adult human case. Some of the drugs appears to repair the SQT1 mutation quite well but not as well as the optimal combination of two drugs. FIG. 9 shows that for the optimal dose of ivabradine 902, which seemed to almost completely repair the AP and Ca²⁺ transient waveforms of the SQT1 CMs, the maximal upstroke velocity and the conduction velocity differ considerably from the wild type case, explaining the relatively high cost function value obtained for this drug.

Table 4 below shows data that provides further basis for evaluating the efficacy of the two drug combinations. Biomarkers computed for the normal state (wild type) and abnormal state (SQT1) adult human ventricular CM cases, are listed as well as for the treated state (SQT1 case with the optimal combination of two drugs) and for the optimal dose of each single drug applied. Note that the combination drug (treated state) repairs all the considered biomarkers in the SQT1 case from deviating up to 35% from the wild type case, to only deviating up to 3% from the wild type case.

TABLE 4
Biomarkers and cost function values

| | | APD50 | | APD90 | | dvdt ₘₐₓ | | CV | | QT | |
|---|---|---|---|---|---|---|---|---|---|---|---|
| | Cost | | % from | | % from | | % from | | % from | | % from |
| | function | ms | WT | ms | WT | mV/ms | WT | cm/s | WT | ms | WT |
| WT (no drug) | 0 | 223 | | 272 | | 215 | | 54 | | 283 | |
| SQT1 (no | 9.3 | 146 | −35% | 189 | −31% | 216 | +1% | 54 | +0% | 203 | −28% |
| drug) | | | | | | | | | | | |
| Combination | 0.2 | 224 | +1% | 276 | +1% | 214 | −0% | 53 | −1% | 274 | −3% |
| drug | | | | | | | | | | | |
| Veratridine | 2.7 | 200 | −10% | 248 | −9% | 215 | +0% | 53 | −1% | 244 | −14% |
| BAY K 8644 | 3.3 | 167 | −25% | 207 | −24% | 214 | −0% | 53 | −0% | 208 | −27% |
| Ivabradine | 3.3 | 223 | −0% | 270 | −1% | 133 | −38% | 44 | −18% | 278 | −2% |
| Disopyramide | 3.9 | 209 | −7% | 259 | −5% | 191 | −11% | 51 | −5% | 264 | −7% |
| Quinidine | 5.2 | 206 | −8% | 255 | −6% | 167 | −22% | 48 | −10% | 261 | −8% |
| Amiodarone | 8.9 | 154 | −31% | 199 | −27% | 211 | −2% | 53 | −1% | 214 | −25% |
| Propafenone | 9.0 | 151 | −32% | 196 | −28% | 212 | −1% | 53 | −1% | 203 | −28% |
| Mexiletine | 9.2 | 157 | −30% | 201 | −26% | 172 | −20% | 49 | −9% | 215 | −24% |
| Ajmaline | 9.3 | 146 | −35% | 189 | −31% | 216 | +1% | 54 | +0% | 203 | −28% |

Table 4: Cost function values and biomarkers of the adult human ventricular CM models for wild type and SQT1 with no drugs present, as well as for the SQT1 model with the optimal combination of two drugs or the optimal dose of the individual drugs applied. The cost function value (see (16)), the action potential durations, APD50 and APD90, the maximal upstroke velocity of the action potential, dvdt max, the conduction velocity, CV, and the QT interval are listed. In the SQT1 cases, we also report the percent difference from the wild type case.

3. Treated State with Low Drug Doses

Whether a single drug, or drug combination, repairs an AP is not the sole consideration when identifying an optimal drug treatment. Drug side effects can also be a concern when developing a multi-compound therapy. By limiting the maximum concentration of individual drugs in a multi-compound therapy it may be possible to produce treatments with fewer side effects.

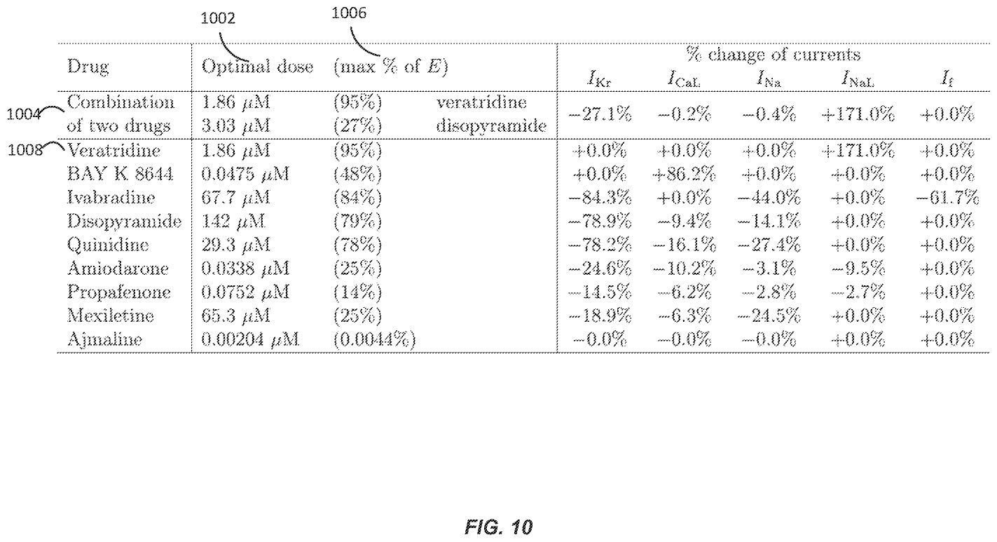

FIG. 10 shows the optimal doses 1002 determined for each drug and for the optimal combination of two drugs 1004 in the adult human case. FIG. 10 also reports the associated block, or increase as a percentage for the individual currents. In addition, we report the effect of the optimal doses in the form of the percentage of the maximum effect (E 1006, see 5) of the drug, for the current most prominently affected by the drug.

FIG. 10 shows that for single drugs and two drug combinations the identified doses 1002 are quite high. For instance in FIG. 10, the optimal dose of veratridine 1008 (1.86 μM) is more than four times higher than the EC₅₀ value of veratridine. The high dosage results in an effect on the I_{NaL} current that is 95% of the maximum effect of veratridine 1008.

FIG. 10 shows that for a 1.86 μM dose of veratridine 430, the effect on I_{NaL} 432 is increased by a factor of 1.8. This is the maximal dose allowed in these applications of the computational procedure. In order to avoid potential side effects of high drug doses, it can be generally beneficial to avoid such large doses. Therefore, the computational procedure can be applied to find optimal drug combinations with lower drug doses.

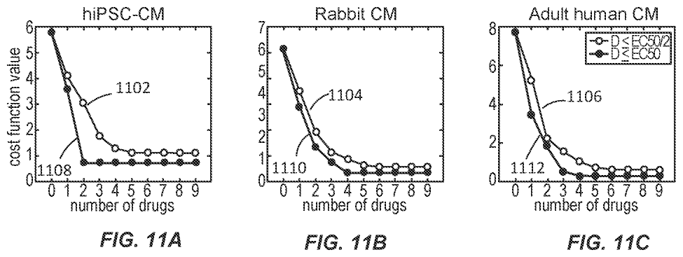

FIGS. 11A-11C shows the optimal cost function values found in the search for optimal drug combinations for an increasing number of drugs, combined with a strict limit on the maximal allowed drug doses. More specifically, the restrictions D≤min(EC₅₀)/2 1102-1106 and D≤min(EC₅₀) 1108-1102 are considered. For the restriction D≤min(EC₅₀)/2 1102-1106, the cost function value is drastically decreased when only one or two drugs are applied, and then gradually decreased until 5-6 drugs are included. Furthermore, for the less strict restriction D≤min(EC₅₀) 1108-1112, fewer drugs are needed to reduce the cost function value. In the hiPSC-CM case, shown in FIG. 11A, it seems like 2 drugs are sufficient to achieve an optimal solution. For the rabbit, shown in FIG. 11B, and adult human cases, depicted in FIG. 11C, 4 drugs seem to provide an effective ‘repair’ of the AP and calcium waveforms.

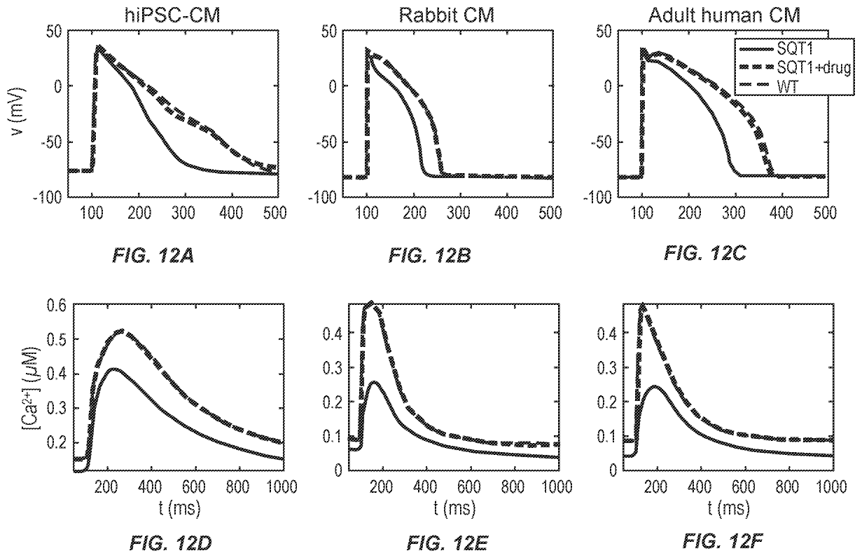

FIGS. 12A-12F shows a plot of the AP in FIGS. 12A-12C and Ca²⁺ transient in FIGS. 11D-11F for the treated state with optimal combinations of 5 drugs with the restriction D≤min(EC₅₀)/2.

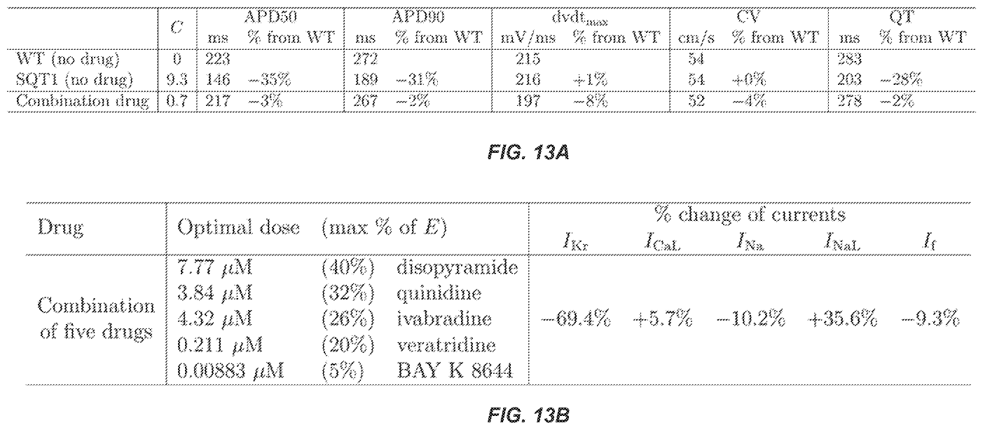

FIGS. 13A and 13B show biomarkers of the solutions and the treated state with optimal drug doses for the adult human case. The combination of 5 drugs with the restriction D≤min(EC₅₀)/2 on the doses seem to be able to repair the AP and Ca²⁺ transient of the SQT1 CMs quite well.

Accordingly the computational methods presented herein can be used to identify multi-compound therapies that can ‘repair’ an abnormal state caused by a mutation. The method is based on information of how a collection of drugs affects the relevant ion channels. For the SQT1 mutation N588K, and the resulting increase of the I_Kr current, the methods were able to identify a theoretical ‘drug’ that completely repair the effect of this mutation as judged by a set of biomarkers. If relatively high drug doses can be utilized, the mutation abnormal state can be repaired using only two drugs. If low doses are required, more individual drugs need to be applied in order to completely repair the mutation abnormal state.

### V. Discussion

There is an unmet need for developing new anti-arrhythmic drugs (see, e.g., [6, 50, 10]) for a whole series of cardiac related conditions. The scientific and regulatory path required for approval of a new compound are, however, both long and extremely costly [51, 52]. These challenges motivate the search for alternatives, and one plausible approach is to search for combinations of existing drugs. Although this sounds like a simple, and straightforward idea to test in a laboratory, the combination of a large group of different drugs applying a range of different drug concentrations quickly becomes a challenging endeavour. In addition, even if such lab experiments were conducted, the end result would be the right compound for the animal cells or hiPSC-CMs under consideration, and not actually a compound suited for adult humans. Using mathematical models, this changes. In principle we are in position to use mathematical models to identify a precise mixed compound for normalizing the AP and thus stabilizing adult human CMs. Since we can also compute the ideal compounds for hiPSC-CMs and rabbit CMs, the suggested drug can be tested in order to gain insight into its applicability.

Our main aim in this study is to use models of the effect of well characterized existing drugs to find optimal combinations of these drugs that repair the effect of a given mutation. Specifically, we need information about how the drugs affect the ion currents governing the AP of the wild type and mutant cells. Here, we have provided an example of how a small collection of known drugs can be combined to ‘define’ a drug that, in simulations, almost completely repairs the AP properties of the mutant myocytes. Our results are founded on measured properties of the drugs under consideration, but our computational endpoints are purely theoretical in the sense that the resulting drug has not been tested in the lab. However, the results are specified in a way that enables laboratory testing. In this section, we will summarize the method, point to possible applications and discuss limitations and possible weaknesses.

The results show the computational methods can identify combined drugs that can ‘repair’ the effects of a mutation. Embodiments can use information of how a collection of drugs affects the relevant ion channels. For the SQT1 mutation N588K, and the resulting increase of the I_{Kr} current, we were able to identify a theoretical ‘drug’ that completely repair the effect of this mutation as judged by a set of biomarkers. If relatively high drug doses can be utilized, the effect of the mutation can be repaired using only two drugs. If low doses are required, more individual drugs need to be applied in order to completely repair the effect of this mutation.

A. Pharmaceutical Considerations

As shown in FIGS. 8A-8F and FIG. 5, one of the key insights from our computational approach for ‘correcting’ the dramatically shortened APD and depressed intracellular Ca²⁺ transient that are both hallmark features of the SQT1 Syndrome is that a combination of two different approved cardiac drugs can be very effective. The electrophysiological principles that underpin this finding are worth reviewing. Veratridine, one of the compounds or drugs, that we have identified as being essential for restoring the APD waveform acts mainly by increasing the amplitude of one particular transmembrane current: the slowly inactivating or late Na⁺ current, I_{NaL} [76]. This small inward current can produce very significant changes in the plateau of the action potential for a number of different reasons. First, although I_{NaL} is small the membrane resistance at the plateau of the AP is relatively high (approximately three times larger than at the resting membrane potential). Second, I_{NaL} shows very little voltage-dependent inactivation and therefore provides an almost constant depolarizing influence over a broad range of membrane potentials [77]. In essence, therefore, I_{NaL} functions very similarly to the effects of the relatively long applied stimulus currents that were used by Wood, Heppner and Weidmann [78] in their original classical demonstration of the effects of plateau height and duration of the action potential waveform on ventricular contractility.

Our quite broadly based survey and related analyses of approved drugs that may be effective in restoring the dramatically shortened action potential that is characteristic of the SQT1 Syndrome, and is caused by a mutation-induced, very marked enhancement of the K⁺ current, I_{Kr}, has identified disopyramide as an effective antidote; and a potent component of a drug combination that can restore the ventricular AP waveform. Once again, this finding has an established functional basis. Perhaps the primary reason for the effectiveness of disopyramide at the concentrations identified as being effective by our computational analysis, this drug potently blocks I_{Kr} (FIG. 5). In addition, since the initiation of repolarization of the mammalian ventricular action potential is known to be regenerative (that is it exhibits all-or-none behaviour), the dynamics of the size of I_{Kr} as well as its average amplitude are critical for initiating the final repolarization phase of the action potential (cf. [79]). Specifically, the blocking actions of disopyramide can significantly reduce the transient increase in the outward component of I_{Kr} that is produced during the final phase of repolarization due to the intrinsic inwardly rectifying property of this particular time- and voltage-dependent K⁺ conductance. In summary, interacting effects of enhancement of I_{NaL} and separate synergistic block of I_{Kr} can dramatically lengthen APD and restore the intracellular Ca²⁺ transient to its control or baseline contour [80]. It is also well known that even small changes in the rates of repolarization of the action potential can significantly alter the intracellular Ca²⁺ transient and associated ventricular contractility [81, 82].

B. Method for Finding Optimal, Combined Compounds

We use the method outlined above to find candidate combinations of known drugs that are effective in repairing the effect of the SQT1 mutation in three cases: hiPSC-CMs, rabbit CMs and adult human CMs. Note, however, that the procedure introduced here can similarly be applied to other mutations. Suppose a mutation changes the dynamics of one (or several) currents. The aim is then to find an optimal combination of a collection of K existing drugs that can ‘repair’ the effect of the mutation. The information needed to apply the method described above is how all K drugs affect the ion currents of the mutant myocytes. Here, we have used simple IC₅₀/EC₅₀ models to represent the effect of the drugs on the individual ion currents. In addition, an accurate AP model of each type mutant myocyte is needed. With this information, we can run simulations to identify an optimal drug that can repair the AP of the mutant myocyte, as judged by alignment with the quantitative biomarkers for the AP waveform and the calcium transient of a wild type myocyte. An feature of our method is that both the set of known drugs, the set of biomarkers, and the model of the effect of the drug, can be changed to address additional goals.

C. The Requirement for Adequate Data Sets

Here, we have considered the SQT1 syndrome and the main reason for this is the availability of data on how combinations of drugs affects the mutated I_{Kr} current; see [28, 29, 30]. We also need information on how the drugs affect all the ion currents of the mutant myocyte that are unaffected by the mutation, but that is more generally available; see, e.g., [45, 46, 53, 54, 55, 56, 57]. With access to data on how a collection of drugs act on other mutations, it is possible to repeat the steps we have taken here to devise optimal, theoretical, drugs for repairing the AP properties of the mutant cells.

D. The Optimal Drug Combination

As shown in FIGS. 7A-7C and illustrated in FIGS. 8A-8F, our analysis reveals that the combination of two drugs can almost completely repair the effect of the mutation on the AP waveform. However, this comes with price of relatively high doses, which are generally not clinically applicable due to off-target interactions and their resulting side effects. From FIG. 11 we see that the doses can be significantly reduced if we allow several (more than two) drugs in the combination. In fact, by requiring that all drugs have concentration below 50% of their lowest EC₅₀, we can completely repair the effect of the mutation using a combination five drugs; see FIGS. 12A-12F.

E. Modeling the Effect of a Drug

Modeling the effects of various drugs in the setting of cardiac arrhythmia has received considerable attention and a general introduction is provided in [58]. The most common approaches to modeling the effect of drugs on ion currents of CMs are based on Markov models and IC₅₀-models. Markov models (see, e.g., [21, 59, 27, 60, 61]) are clearly more detailed and, at least in some cases, closely tied to the molecular composition and biophysical properties of the channel. The disadvantage is that these models require very detailed data on every individual current and such data are often not available. Ideally, in order to parameterize a Markov model properly, data from single channel measurements should be used (see, e.g., [62, 63, 64, 65]), and such data are not commonly available. In contrast, the IC₅₀-type modeling that we have used (see, e.g., [66, 67, 68]) is much simpler and can be estimated based on few biomarkers (see, e.g., [33]). In the Supplementary information, we give an example where we compare an IC₅₀ model with a comprehensive Markov model from [69]. We have used this specific case because the Markov model from [69] is completely specified with all necessary parameters. FIGS. 2A-2B show that these two ion channel model alternatives give similar results. In fact we prefer to use accurate models based on Markov models, but the necessary data is not presently available.

As mentioned above, we assume that the effect of two drugs can be approximated by multiplying the individual effects of the two drugs. The justification of this approximation is as follows: Suppose the open probability of a certain ion channel is given by o. If we apply a blocker referred to as A to this channel, we assume that a certain fraction, μ_{A}≤1, of the channels will be blocked (see 7). After the application of this drug, the open probability of the channel is μ_{A}o. Next, we assume that we have another drug, denoted by B. This drug is also a blocker and it blocks a fraction μ_{B}≤1 of the open channels. By first applying the drug A, the open probability is μ_{A}o, and then, by applying the drug B the open probability becomes μ_{B}μ_{A}o. Thus, we have assumed that the binding of the two blockers is non-interactive (strictly independent), i.e., they neither compete nor allosterically facilitate each others binding. In the Supplementary information, we further discuss the question concerning the multiplicative effect of drug compounds. Using recent measurements from [70] we indicate that the effect of two blockers can be approximated by multiplying the effect of the two drugs.

It should also be noted the IC₅₀ values reported in the literature can vary significantly. In [71, 72] it is argued that the reason for these differences may be the lack of uniformly accepted comprehensive protocols for measuring IC₅₀ values.

F. Previous Attempts to Utilize Anti-Arrhythmic Drug Combinations

The concept of combining two drugs in clinical cardiac electrophysiology in order to achieve advantageous (anti-arrhythmic) outcomes has been evaluated in both animal studies and clinical settings; see e.g. [73, 74, 75]. However, this approach seems to have received relatively little attention during the past 15 years. These earlier papers on combined drugs, usually give the effect in terms of clinical characteristics that are difficult to use in order to evaluate our hypothesis of multiplicative blocks (see 9).

G. Supplementary Inversion Results

In this section, some supplementary results from the computational procedure are reported.

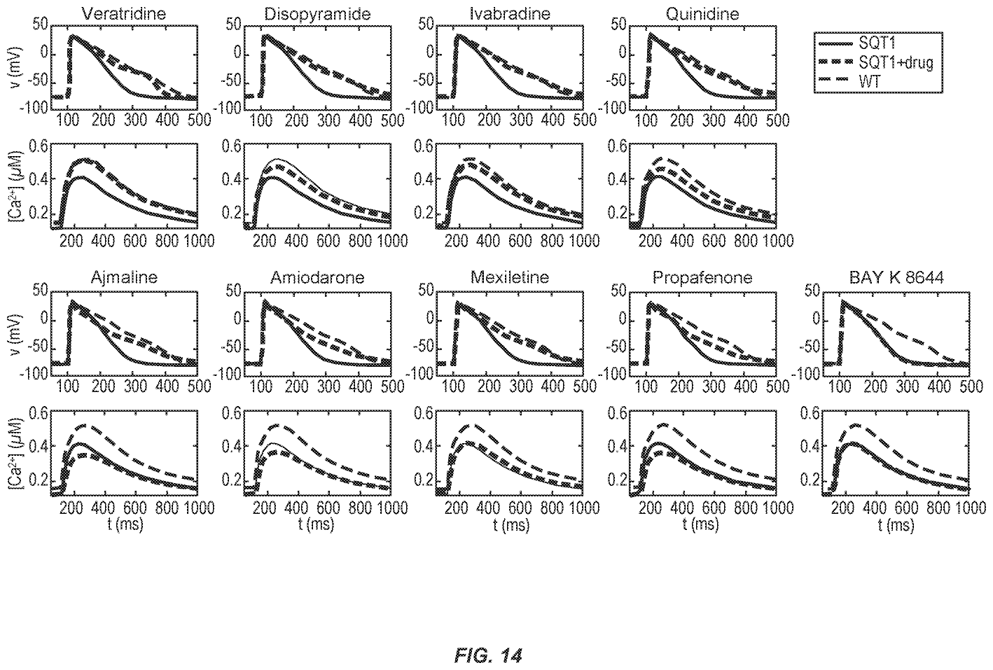

FIG. 14 shows, the AP and Ca²⁺ transients of the SQT1 models following application of optimal doses of a single drug for repairing the SQT1 mutation in hiPSC-CMs are plotted and compared to the wild type and SQT1 AP and Ca²⁺ transients with no drug applied.

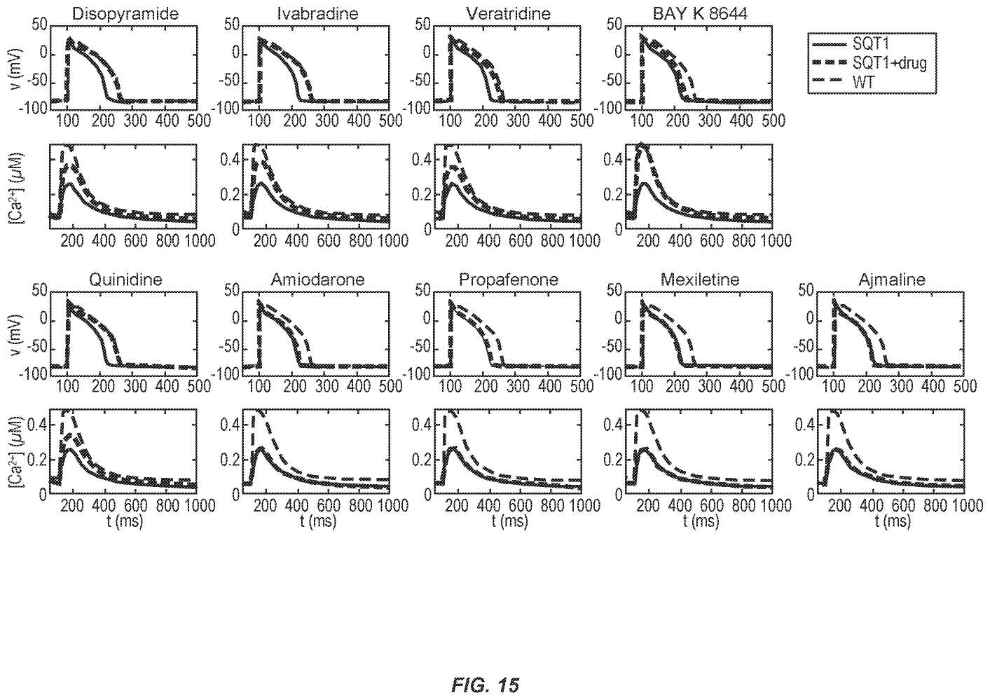

FIG. 15 shows the AP and Ca²⁺ transients of the SQT1 models following application of optimal doses of a single drug for repairing the SQT1 mutation in and rabbit CMs are plotted and compared to the wild type and SQT1 AP and Ca²⁺ transients with no drug applied.

Tables 5 and 6 report the value of a number of biomarkers from the wild type and SQT1 models, in addition to the SQT1 cases after optimal drug doses is applied. Tables 7 and 8 report the optimal doses found in each case.

Furthermore, Tables 9 and 10 report biomarkers for the SQT1 models for hiPSC-CMs and rabbit ventricular CMs with the optimal combination of five drugs with the restriction D≤min(EC₅₀)/2 applied, and Tables 11 and 12 report the optimal drug doses in these optimal drug combinations.

TABLE 5

| | Cost | APD50 | | APD90 | | dvdt ₘₐₓ | | CV | |
|---|---|---|---|---|---|---|---|---|---|
| | function | ms | % from WT | ms | % from WT | mV/ms | % from WT | cm/s | % from WT |
| WT (no drug) | 0 | 177 | | 325 | | 31 | | 5 | |
| SQT1 (no drug) | 8.1 | 106 | −40% | 188 | −42% | 32 | +3% | 5 | +1% |
| Combination drug | 0.6 | 160 | −10% | 325 | −0% | 31 | −0% | 5 | −2% |
| Veratridine | 1.8 | 151 | −15% | 288 | −12% | 32 | +4% | 5 | −1% |
| Disopyramide | 2.1 | 153 | −13% | 348 | +7% | 26 | −14% | 4 | −8% |
| Ivabradine | 2.8 | 156 | −12% | 346 | +6% | 21 | −31% | 4 | −17% |
| Quinidine | 2.8 | 148 | −16% | 329 | +1% | 24 | −22% | 4 | −11% |
| Ajmaline | 3.8 | 140 | −21% | 325 | −0% | 26 | −14% | 4 | −8% |
| Amiodarone | 5.0 | 131 | −26% | 319 | −2% | 21 | −30% | 4 | −16% |
| Mexiletine | 5.5 | 145 | −18% | 325 | −0% | 10 | −66% | 3 | −41% |
| Propafenone | 5.7 | 127 | −28% | 302 | −7% | 21 | −31% | 4 | −16% |
| BAY K 8644 | 5.8 | 106 | −40% | 188 | −42% | 32 | +3% | 5 | +1% |

Table 5 shows cost function value and biomarkers of the hiPSC-CM models for wild type and SQT1 with no drugs present, as well as for the SQT1 model with the optimal combination of two drugs or the optimal dose of the individual drugs applied. We report the cost function value, the action potential durations, APD50 and APD90, the maximal upstroke velocity of the action potential, dvdt max, and the conduction velocity, CV. In the SQT1 cases, we also report the percent difference from the wild type case.

TABLE 6

| | Cost | APD50 | | APD90 | | dvdt ₘₐₓ | | CV | | QT | |
|---|---|---|---|---|---|---|---|---|---|---|---|
| | function | ms | % from WT | ms | % from WT | mV/ms | % from WT | cm/s | % fromWT | ms | % from WT |
| WT (no drug) | 0 | 133 | | 157 | | 156 | | 45 | | 163 | |
| SQT1 (no drug) | 7.3 | 94 | −29% | 117 | −25% | 157 | +0% | 45 | +0% | 132 | −19% |
| Combination drug | 0.4 | 135 | +1% | 160 | +2% | 153 | −2% | 44 | −2% | 153 | −6% |
| Disopyramide | 2.3 | 132 | −0% | 157 | +0% | 140 | −11% | 43 | −5% | 166 | +2% |
| Ivabradine | 2.6 | 133 | +0% | 157 | +0% | 114 | −27% | 39 | −14% | 164 | +1% |
| Veratridine | 3.0 | 121 | −9% | 145 | −7% | 156 | +0% | 45 | −1% | 142 | −13% |
| BAY K 8644 | 3.0 | 106 | −20% | 129 | −18% | 156 | +0% | 45 | −1% | 121 | −26% |
| Quinidine | 3.4 | 132 | −1% | 156 | −1% | 119 | −24% | 40 | −12% | 168 | +3% |
| Amiodarone | 6.7 | 101 | −24% | 124 | −21% | 151 | −3% | 44 | −1% | 139 | −14% |
| Propafenone | 6.9 | 99 | −25% | 123 | −22% | 151 | −3% | 44 | −2% | 138 | −15% |
| Mexiletine | 7.3 | 95 | −28% | 118 | −25% | 153 | −2% | 45 | −1% | 133 | −18% |
| Ajmaline | 7.3 | 94 | −29% | 117 | −25% | 157 | +0% | 45 | +0% | 132 | −19% |

Table 6 shows cost function value and biomarkers of the rabbit ventricular CM models for wild type and SQT1 with no drugs present, as well as for the SQT1 model with the optimal combination of two drugs or the optimal dose of the individual drugs applied. We report the cost function value, the action potential durations, APD50 and APD90, the maximal upstroke velocity of the action potential, dvdt max, the conduction velocity, CV, and the QT interval. In the SQT1 cases, we also report the percent difference from the wild type case.

TABLE 7

| | Optimal | | | | % change of currents | | | | |
|---|---|---|---|---|---|---|---|---|---|
| Drug | dose | | (max % of E) | | I_{Kr} | I_{CaL} | I_{Na} | I_{NaL} | I_{f} |
| Combination | 0.502 | μM | (58%) | veratridine | 2* − 41.1% | 2* − 0.6% | 2* − 1.2% | 2* + 104.7% | 2* + 0.0% |
| of two drugs | 8.63 | μM | (41%) | disopyramide | | | | | |
| Veratridine | 1.86 | μM | (95%) | | +0.0% | +0.0% | +0.0% | +171.0% | +0.0% |
| Disopyramide | 131 | μM | (78%) | | −78.1% | −8.7% | −13.4% | +0.0% | +0.0% |
| Ivabradine | 42.6 | μM | (77%) | | −77.2% | +0.0% | −33.0% | +0.0% | −50.3% |
| Quinidine | 22.1 | μM | (73%) | | −73.1% | −12.6% | −22.1% | +0.0% | +0.0% |
| Ajmaline | 73 | μM | (61%) | | −51.2% | −61.0% | −14.4% | +0.0% | +0.0% |
| Amiodarone | 1.41 | μM | (68%) | | −67.8% | −51.5% | −30.5% | −31.9% | +0.0% |
| Mexiletine | 444 | μM | (69%) | | −61.2% | −31.5% | −68.8% | +0.0% | +0.0% |
| Propafenone | 1.65 | μM | (60%) | | −59.6% | −51.4% | −31.7% | −30.9% | +0.0% |
| BAY K 8644 | 0.000282 | μM | (0.015%) | | +0.0% | +0.0% | +0.0% | +0.0% | +0.0% |

Table 7 shows optimal doses and associated change of currents found for single drugs or a combination of two drugs for repairing the SQT1 mutation in hiPSC-CMs.

TABLE 8

| | Optimal | | | | % change of currents | | | | |
|---|---|---|---|---|---|---|---|---|---|
| Drug | dose | | (max % of E) | | I_{Kr} | I_{CaL} | I_{Na} | I_{NaL} | I_{f} |
| Combination | 1.86 | μM | (95%) | veratridine | 2* − 49.6% | 2* − 1.1% | 2* − 2.1% | 2* + 171.0% | 2* + 0.0% |
| of two drugs | 15.3 | μM | (50%) | disopyramide | | | | | |
| Disopyramide | 122 | μM | (77%) | | −77.3% | −8.2% | −12.8% | +0.0% | +0.0% |
| Ivabradine | 38.5 | μM | (75%) | | −75.3% | +0.0% | −30.9% | +0.0% | −47.8% |
| Veratridine | 1.86 | μM | (95%) | | +0.0% | +0.0% | +0.0% | +171.0% | +0.0% |
| BAY K 8644 | 0.0642 | μM | (60%) | | +0.0% | +108.9% | +0.0% | +0.0% | +0.0% |
| Quinidine | 29.7 | μM | (78%) | | −78.5% | −16.2% | −27.6% | +0.0% | +0.0% |
| Amiodarone | 0.0492 | μM | (28%) | | −28.2% | −12.4% | −4.0% | −10.9% | +0.0% |
| Propafenone | 0.131 | μM | (20%) | | −20.0% | −9.7% | −4.5% | −4.4% | +0.0% |
| Mexiletine | 4.97 | μM | (2.4%) | | −1.7% | −0.5% | −2.4% | +0.0% | +0.0% |
| Ajmaline | 8.88e−16 | μM | (0%) | | +0.0% | +0.0% | +0.0% | +0.0% | +0.0% |

Table 8 shows optimal doses and associated change of currents found for single drugs or a combination of two drugs for repairing the SQT1 mutation in rabbit ventricular CMs.

TABLE 9

| | Cost | APD50 | | APD90 | | dvdt ₘₐₓ | | CV | |
|---|---|---|---|---|---|---|---|---|---|
| | function | ms | % from WT | ms | % from WT | mV/ms | % from WT | cm/s | % from WT |
| WT (no drug) | 0 | 177 | | 325 | | 31 | | 5 | |
| SQT1 (no drug) | 8.1 | 106 | −40% | 188 | −42% | 32 | +3% | 5 | +1% |
| Combination drug | 1.1 | 156 | −12% | 325 | −0% | 28 | −8% | 5 | −5% |

Table 9 shows cost function value and biomarkers for the SQT1 hiPSC-CM model with the optimal combination of five drugs with the restriction D≤min(EC₅₀)/2 applied.

TABLE 10

| | | APD50 | | APD90 | | dvdt ₘₐₓ | | CV | | QT | |
|---|---|---|---|---|---|---|---|---|---|---|---|
| | C | ms | % from WT | ms | % from WT | mV/ms | % from WT | cm/s | % from WT | ms | % from WT |
| WT (no drug) | 0 | 133 | | 157 | | 156 | | 45 | | 163 | |
| SQT1 (no drug) | 7.3 | 94 | −29% | 117 | −25% | 157 | +0% | 45 | +0% | 132 | −19% |
| Combination drug | 0.8 | 130 | −2% | 155 | −1% | 140 | −10% | 43 | −5% | 156 | −4% |

Table 10 shows cost function value and biomarkers for the SQT1 rabbit ventricular model with the optimal combination of five drugs with the restriction D≤min(EC₅₀)/2 applied.

TABLE 11

| | | | | % change of currents | | | | |
|---|---|---|---|---|---|---|---|---|
| Drug | Optimal dose | (max % of E) | | I_{Kr} | I_{CaL} | I_{Na} | I_{NaL} | I_{f} |
| Combination | 7.63 μM | (39%) | disopyramide | −66.7% | +11.2% | −8.9% | +35.9% | −7.0% |
| of five drugs | 3.71 μM | (31%) | quinidine | | | | | |
| | 0.213 μM | (20%) | veratridine | | | | | |
| | 3.16 μM | (20%) | ivabradine | | | | | |
| | 0.012 μM | (8.1%) | BAY K 8644 | | | | | |

Table 11 shows optimal doses of a combination of five drugs with the restriction D≤min(EC₅₀)/2 found for repairing the SQT1 mutation in hiPSC-CMs.

TABLE 12

| | | | | % change of currents | | | | |
|---|---|---|---|---|---|---|---|---|
| Drug | Optimal dose | (max % of E) | | I_{Kr} | I_{CaL} | I_{Na} | I_{NaL} | I_{f} |
| Combination | 7.85 μM | (40%) | disopyramide | 5* − 73.0% | 5* + 9.6% | 5* − 12.3% | 5* + 35.4% | 5* − 12.9% |
| of five drugs | 4.03 μM | (33%) | quinidine | | | | | |
| | 6.21 μM | (33%) | ivabradine | | | | | |
| | 0.211 μM | (20%) | veratridine | | | | | |
| | 0.0112 μM | (7.3%) | BAY K 8644 | | | | | |

Table 12 shows optimal doses of a combination of five drugs with the restriction D; min(EC₅₀)/2 found for repairing the SQT1 mutation in rabbit ventricular CMs.

### VI. Method for Identifying Viable Treatment

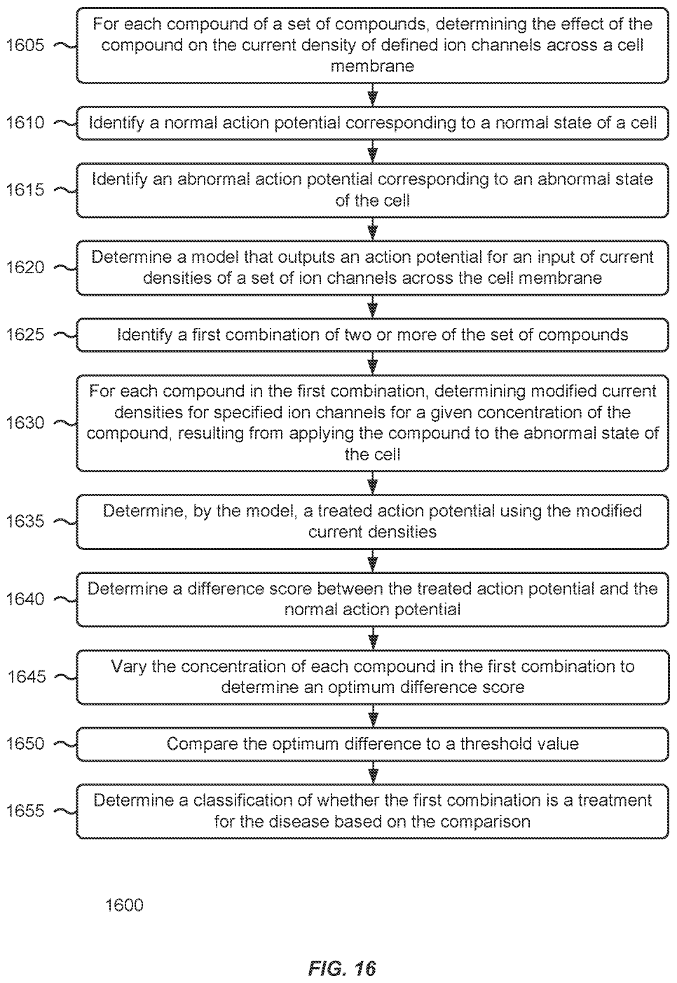

FIG. 16 is a flowchart of an example process 1600 for developing a multi-compound therapy to repair an action potential. Process 1600 may be performed by a computer system, which may take various forms, e.g., a server computer, a laptop computer, a mobile device, etc.

At block 1605, the effect of the compound on the current density of defined ion channels across a cell membrane is determined for each compound of a set of compounds. The cell membrane can be for any type of cell with an action potential including cardiac cells, nerve cells, muscle cells, or endocrine cells. The effect of the compound on the current densities of specified ion channels can be determined by retrieving data from a compound database.

At block 1610, a normal action potential corresponding to a normal state of a cell can be identified. A normal action potential can be identified using in silico, in vitro (e.g., subcellular patch-clamp), or in vivo methods. Some methods, such as genetically encoded calcium indicators (GECI, e.g., GCaMP), can be used in vitro and in vivo. The action potential can include an action potential duration, a maximal upstroke velocity, and an action potential amplitude. The action potential can also include an ion transient duration, a maximal ion transient upstroke velocity, and an ion transient amplitude. In some implementations, the normal state of the cell is a wild type.

At block 1615, an abnormal action potential corresponding to an abnormal state of the cell can be identified. The abnormal state can be determined for diseases with documented changes to the cellular ion channels. The abnormal state could be determined through analysis of a tissue sample taken from a patient with the disease. The cell can be a neuron and the diseases can include epilepsy, episodic ataxia, familial hemiplegic migraine, Lambert-Eaton myasthenic syndrome, Alzheimer's disease, Parkinson's disease, schizophrenia, or hyperekplexia. The cell can be a cardiac cell and the diseases can include Brugada syndrome, long QT-syndrome, short QT-syndrome, atrial standstill, or sick sinus syndrome.

At block 1620, a model is determined that outputs an action potential for an input of current densities of a set of ion channels across the cell membrane. For example, a model for an action potential is discussed in section I.B.

At block 1625, a first combination of two or more of the set of compounds is determined. If the first combination is not found to be a treatment for the disease, alternative combinations of two compounds are tried until a treatment for the disease is identified. If no combination of two compounds is identified as a treatment for the disease, larger combinations of compounds can be determined (e.g., three compound combinations). A combination of compounds, such as the first combination, can comprise at least any of the following number of compounds: 1, 2, 3, 4, 5, 6, 7, 8, 9, 10, 11, 12, 13, 14, 15, 20, 30, 40, 50, 60, 70, 80, 90, 100, or more.

At block 1630, the modified current densities for specified ion channels for a given concentration of the compound can be determined for each compound in the first combination by applying the compound to the abnormal state of the cell. For example, the modified current densities for a given concentration of the compound can be determined by inputting the compound concentration to an IC₅₀/EC₅₀ model.

At block 1635, the modified current densities can be used to determine a treated action potential. Determining the treated action potential can include multiplying the current density in the abnormal model by the effect of each compound in the first combination on the current density. For example, a model for the effect of multiple compounds on an action potential is discussed in section II.B.

At block 1640, a difference score between the treated action potential and the normal action potential is determined for the first combination. The difference score can be a value of a cost function such as the cost function discussed in section III.

At block 1645, process 1600 may include varying the concentration of each compound in the first combination to determine an optimum difference score. In some implementations, the concentration of each compound is constrained so as to not exceed a concentration threshold as discussed in section IV.D.3. The optimum difference score can be a value of the minimized cost function. The concentration of compounds can be any of the following molar concentrations (mol/L): 1000, 100, 10, 1, 0.1, 0.01, 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, 10⁻¹⁸, 10⁻¹⁹, 10⁻²⁰, 10⁻²¹, 10⁻²², 10⁻²³, 10⁻²⁴, 10⁻²⁵, 10⁻²⁶, etc. The concentration of compounds that produces the optimum difference score can be the optimum concentration of compounds.

At block 1650, the optimum difference can be compared to a threshold value. The threshold value can correspond to a minimum percentage similarity to a normal action potential, i.e., minimum that is acceptable. As examples, a minimum percentage similarity can be 0.5%, 1%, 2%, 3%, 4%, 5%, 6%, 7%, 8%, 9%, 10%, 11%, 12%, 13%, 14%, 15%, 16%, 17%, 18%, 20%, etc.

At block 1655, a classification of whether the first combination is a treatment for the disease can be determined based on the comparison. A drug can be created that contains the first combination of compounds. The concentration of each compound in the drug can vary from the optimum concentration for that compound by 0.1%, 0.5%, 1%, 2%, 3%, 4%, 5%, 6%, 7%, 8%, 9%, 10%, 11%, 12%, 13%, 14%, 15%, 16%, 17%, 18%, 20%, etc. A patient with the disease can be treated with the first combination of compounds. The first combination can be classified as a treatment of the disease if the optimum difference is below a threshold value.

Process 1600 may include additional implementations, such as any single implementation or any combination of implementations described below and/or in connection with one or more other processes described elsewhere herein.

Although FIG. 16 shows example blocks of process 1600, in some implementations, process 1500 may include additional blocks, fewer blocks, different blocks, or differently arranged blocks than those depicted in FIG. 16. Additionally, or alternatively, two or more of the blocks of process 1600 may be performed in parallel.

### VII. Computer System

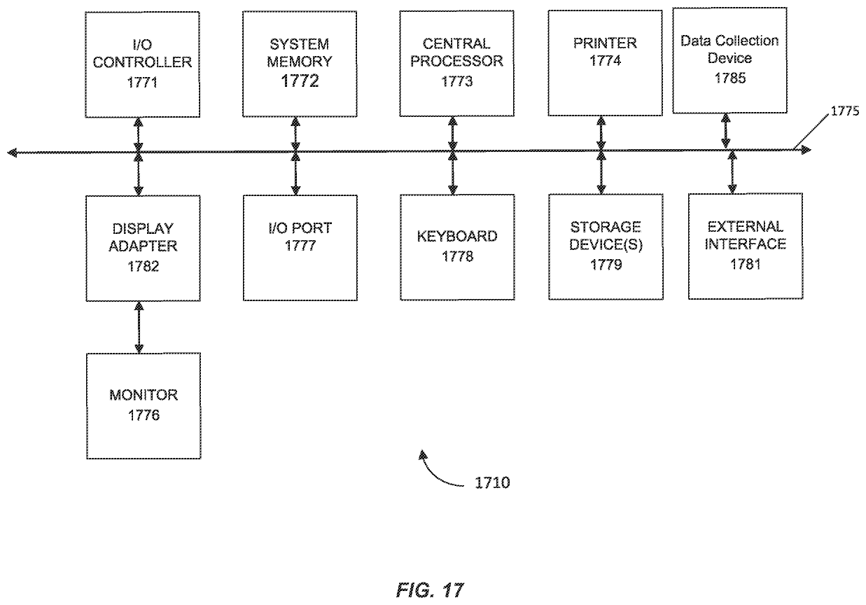

Any of the computer systems mentioned herein may utilize any suitable number of subsystems. Examples of such subsystems are shown in FIG. 17 in computer system 1710. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 17 are interconnected via a system bus 1775. Additional subsystems such as a printer 1774, keyboard 1778, storage device(s) 1779, monitor 1776 (e.g., a display screen, such as an LED), which is coupled to display adapter 1782, and others are shown. Peripherals and input/output (I/O) devices, which couple to I/O controller 1771, can be connected to the computer system by any number of means known in the art such as input/output (I/O) port 1777 (e.g., USB, FireWire©). For example, I/O port 1777 or external interface 1781 (e.g. Ethernet, Wi-Fi, etc.) can be used to connect computer system 1710 to a wide area network such as the Internet, a mouse input device, or a scanner. The interconnection via system bus 1775 allows the central processor 1773 to communicate with each subsystem and to control the execution of a plurality of instructions from system memory 1772 or the storage device(s) 1779 (e.g., a fixed disk, such as a hard drive, or optical disk), as well as the exchange of information between subsystems. The system memory 1772 and/or the storage device(s) 1779 may embody a computer readable medium. Another subsystem is a data collection device 1785, such as a camera, microphone, accelerometer, and the like. Any of the data mentioned herein can be output from one component to another component and can be output to the user.

A computer system can include a plurality of the same components or subsystems, e.g., connected together by external interface 1781, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

### VII. REFERENCES

[1] Umang Patel and Behzad B Pavri, Cardiology in Review, 17(6):300{303, 2009.
[2] Dominic G Whittaker et al., Wiley Interdisciplinary Reviews: Systems Biology and Medicine, 12(4): e1482, 2020.
[3] Hugues Abriel and Elena V Zaklyazminskaya, Gene, 517(1):1{11, 2013.
[4] Anna Fernandez-Falgueras et al., Biology, 6(1):7, 2017.
[5] Zhilin Qu, Gang Hu, Alan Garnkel, and James N Weiss, Physics Reports, 543(2), 2014.
[6] Peter J Schwartz et al., Nature Reviews Disease Primers, 6(1):1-22, 2020.
[7] Fiorenzo Gaita et al., Circulation, 108(8):965-970, 2003.
[8] Ihor Gussak et al., Cardiology, 94(2):99-102, 2000.
[9] Chinmay Patel et al., Circulation: Arrhythmia and Electrophysiology, 3(4):401-408, 2010
[10] Oscar Campuzano et al., Frontiers in Cardiovascular Medicine, 5:149, 2018.
[11] Michelangelo Paci et al., Heart Rhythm, 2017.
[12] Karoline Horgmo Jaeger et al., PLoS Computational Biology, 17(2): e1008089, 2021.
[13] Fiorenzo Gaita et al., Journal of the American College of Cardiology, 43(8):1494-1499, 2004.
[14] Boris Rudic et al., Arrhythmia & electrophysiology review, 3(2):76, 2014.
[15] Andrea Mazzanti et al., Journal of the American College of Cardiology, 63(13):1300-1308, 2014.
[16] Karine Guerrier et al., Circulation: Arrhythmia and Electrophysiology, 8(6):1460-1464, 2015.
[17] T O'Hara et al., PLoS Computational Biology, 7(5): e1002061, 2011.
[18] D C Kernik et al., The Journal of Physiology, 597(17):4533-4564, 2019.
[19] E Grandi et al., Journal of Molecular and Cellular Cardiology, 48(1):112-121, 2010.
[20] Y Rudy and J R Silva, Quarterly Reviews of Biophysics, 39(01):57-116, 2006.
[21] Colleen E. Clancy and Yoram Rudy, Nature, 400:566-569, 1999.
[22] Colleen E. Clancy and Yoram Rudy, Circulation, 105(10):1208-1213, 2002.
[23] Zheng I. Zhu and Colleen E. Clancy, AJP: Heart and Circulatory Physiology, 293(6): H3480-H3489, October 2007.
[24] Colleen E Clancy et al., American Journal of Physiology-Heart and Circulatory Physiology, 292(1): H66-H75, 2007.
[25] Aslak Tveito and Glenn T Lines, Mathematical Biosciences, 217(2):167-173, 2009.
[26] Gary R Mirams, et al., Cardiovascular Research, 91(1):53-61, 2011.
[27] A Tveito and G T Lines, Computing Characterizations ofDrugsfor Ion Channels and Receptors Using Markov Models. Springer-Verlag, Lecture Notes, vol. 111, 2016.
[28] Ibrahim El-Battrawy et al. Journal of the American Heart Association, 7(7): e007394, 2018.
[29] Zhihan Zhao et al., ClinicalPharmacology & Therapeutics, 106(3):642-651, 2019.
[30] M J McPate et al., British Journal of Pharmacology, 155(6):957-966, 2008.
[31] A Tveito et al., Scientific Reports, 8(1):17626, 2018.
[32] Karoline Horgmo Jaeger et al., Frontiers in Pharmacology, 10:1648, 2020.
[33] Aslak Tveito et al., Scientific Reports, 10(1):1-11, 2020.
[34] Mark J McPate et al., Biochemical and Biophysical Research Communications, 334(2):441-449, 2005.
[35] Christian Wolpert et al., Journal of Cardiovascular Electrophysiology, 16(1):54-58, 2005.
[36] Thomas R Shannon et al., Biophysical Journal, 87(5):3351-3371, 2004.
[37] Katja E Odening et al., European Heart Journal, 40(10):842-853, 2019.
[38] Aslak Tveito et al., Frontiers in Physics, 5:48, 2017.
[39] Karoline Horgmo Jaeger et al., PLoS Computational Biology, 15(5): e1007042, 2019.
[40] Karoline Horgmo Jaeger and Aslak Tveito, In Modeling Excitable Tissue, pages 1-13. Springer, Cham, 2020.
[41] R. Anderson et al., Computers & Mathematics with Applications, 2020.
[42] MFEM: Modular finite element methods [Software]. mfem.org.
[43] Karoline Horgmo Jaeger et al., Frontiers in Physics, 8:539, 2021.
[44] Karoline Horgmo Jaeger et al., In Modeling Excitable Tissue, pages 44-55. Springer, Cham, 2020.
[45] J Kramer et al., Scientific Reports, 3:2100, 2013.
[46] W J Crumb et al., Journal of Pharmacological and Toxicological Methods, 81:251-262, 2016. Focused Issue on Safety Pharmacology.
[47] Martin Bechem and Matthias Schramm, Journal of Molecular and Cellular Cardiology, 19:63-75, 1987.
[48] Gunter Thomas et al., Circulation Research, 56(1):87-96, 1985.
[49] Wesley L McKeithan et al., Frontiers in Physiology, 8:766, 2017.
[50] Joachim R Ehrlich and Stanley Nattel et al., Drugs, 69(7):757-774, 2009.
[51] J A DiMasi, H G Grabowski, and R W Hansen, Journal of Health Economics, 47:20-33, 2016.
[52] S M Paul et al., Nature Reviews Drug Discovery, 9(3):203-214, 2010.
[53] Pei-Chi Yang et al., The Journal ofPhysiology, 593(6):1429-1442, 2015.
[54] Pei-Chi Yang et al., Journal ofMolecular and Cellular Cardiology, 99:151-161, 2016.
[55] Earl H Wood et al., Circulation Research, 24(3):409-445, 1969.
[56] Beatriz Trenor et al., The Journal ofPhysiology, 595(21):6599-6612, 2017.
[57] Lin Wu et al., Circulation, 123(16):1713-1720, 2011.
[58] R A Bouchard, R B Clark, and W R Giles., Circulation Research, 76(5):790-801, 1995.
[59] Rajan Sah, Rafael J Ramirez, and Peter H Backx, Circulation research, 90(2):165-173, 2002.
[60] P Orvos et al., Toxicological Sciences, 168(2):365-380, 2019.
[61] Y Qu and H M Vargas, Toxicological Sciences, 147(1):286-295, 2015.
[62] Y Katayama et al., Journal of Pharmacological Sciences, 97, 2005.
[63] Junyi Ma et al., American Journal of Physiology-Heart and Circulatory Physiology, 301(5): H2006-H2017, 2011. PMID: 21890694.
[64] J K Gibson et al., Journal of Pharmacological and Toxicological Methods, 70(3):255-267, 2014.
[65] Panos Macheras and Athanassios Iliadis, Modeling in biopharmaceutics, pharmacokinetics and pharmacodynamics: homogeneous and heterogeneous approaches, volume 30. Springer, 2016.
[66] C E Clancy, Z I Zhu, and Y Rudy, AJP: Heart and Circulatory Physiology, 292(1): H66-H75, 2007.
[67] A Tveito, M M Maleckar, and G T Lines, Computational and Mathematical Biophysics, 6(1):41-64, 2018.
[68] Vladimir Yarov-Yarovoy, Toby W Allen, and Colleen E Clancy, Drug Discovery Today: Disease Models, 14:3-10, 2014.
[69] Feng Qin, Anthony Auerbach, and Frederick Sachs, Biophysical Journal, 70:264-280, January 1996.
[70] Feng Qin, Anthony Auerbach, and Frederick Sachs, Biophysical Journal, 79:1915-1927, January 2000.
[71] Ivo Siekmann et al., BiophysicalJournal, 100(8):1919-1929, April 2011.
[72] Aslak Tveito et al., Mathematical Biosciences, 277:126-135, 2016.
[73] T Brennan, M Fink, and B Rodriguez, European Journal ofPharmaceutical Sciences, 36(1):62-77, 2009.
[74] Mark Ri Davies et al., American Journal of Physiology-Heart and Circulatory Physiology, 2012.
[75] Nejib Zemzemi et al., British Journal ofPharmacology, 168(3):718-733, 2013.
[76] Joachim Almquist, Biophysical Journal, 99(9):2726-2736, 2010.
[77] Gongxin Wang et al., bioRxiv, 2020.
[78] William Lee et al., Molecular Pharmacology, 95(5):537-550, 2019.
[79] Julio Gomis-Tena et al., Journal of Chemical Information andModeling, 60(3):1779-1790, 2020.
[80] Henry J Du et al., Journal of the American College of Cardiology, 10(5):1149-1156, 1987.
[81] Li Wang, Nipavan Chiamvimonvat, and H J Du, Journal of Pharmacology and Experimental Therapeutics, 264(3):1056-1062, 1993.
[82] Henry J Du, Cardiac Electrophysiology Review, 2(2):142-146, 1998.
[83] Karoline Horgmo Jaeger et al., Frontiers in Pharmacology, 11:569489, 2021.
[84] Matthew E Hartman, Dao-Fu Dai, and Michael A Laflamme, Advanced Drug Delivery Reviews, 96:3-17, 2016.
[85] Nikki H L van den Heuvel et al., Journal of Molecular and Cellular Cardiology, 67:12-25, 2014.
[86] Shin Kadota et al., European Heart Journal, 34(15):1147-1156, 2013.
[87] Masahide Kawatou et al., Nature Communications, 8(1):1-11, 2017.
[88] Rami Shinnawi et al., Journal of the American College of Cardiology, 73(18):2310-2324, 2019.
[89] Kazutaka Gima and Yoram Rudy, Circulation Research, 90(8):889-896, 2002.

What is claimed is:

1. A method for multi-compound therapies to correct action potentials in cells associated with a disease, the method comprising:
for each compound of a set of compounds, determining an effect of the compound on a current density of defined ion channels across a cell membrane;
identifying a normal action potential corresponding to a normal state of a cell;
identifying an abnormal action potential corresponding to an abnormal state of the cell;
determining a model that outputs an action potential for an input of current densities of a set of ion channels across the cell membrane;
identifying a first combination of two or more of the set of compounds;
for each compound in the first combination:
determining modified current densities for specified ion channels for a given concentration of the compound, resulting from applying the compound to the abnormal state of the cell;
determining, by the model, a treated action potential using the modified current densities;
determining a difference score between the treated action potential and the normal action potential;
varying the concentration of each compound in the first combination to determine an optimum concentration for each compound in the first combination that results in an optimum difference score;
comparing the optimum difference to a threshold value; and
determining a classification of whether the first combination is a treatment for the disease based on the comparison.

2. The method of claim 1, wherein the concentration of each compound is constrained to not exceed a respective concentration threshold when varying the concentration of each compound.

3. The method of claim 1, further comprising:
creating a drug that includes the first combination at the optimum concentration for each compound.

4. The method of claim 3, further comprising:
treating a patient with the disease using the drug.

5. The method of claim 1, wherein identifying the abnormal action potential corresponding to the abnormal state of the cell includes:
analyzing a patient tissue sample from a patient having the disease.

6. The method of claim 1, wherein determining the effect of the compound includes retrieving data from a compound database.

7. The method of claim 1, wherein determining modified current densities includes inputting the compound concentration to an IC₅₀/EC₅₀ model.

8. The method of claim 1, wherein determining a treated action potential includes multiplying the current density in the abnormal model by the effect of each compound in the first combination on the current density.

9. The method of claim 1, wherein the action potential includes an action potential duration, a maximal upstroke velocity, and an action potential amplitude.

10. The method of claim 1, wherein the action potential includes an ion transient duration, a maximal ion transient upstroke velocity, and a ion transient amplitude.

11. The method of claim 1, wherein the threshold value corresponds to a minimum percentage similarity to the normal action potential.

12. The method of claim 1, wherein the normal state of the cell is a wild type.

13. The method of claim 1, wherein the first combination is classified as a treatment of the disease if the optimum difference is below the threshold value.

14. The method of claim 1, wherein the cells associated with the disease are neurons, and wherein the disease is epilepsy, episodic ataxia, familial hemiplegic migraine, Lambert-Eaton myasthenic syndrome, Alzheimer's disease, Parkinson's disease, schizophrenia, or hyperekplexia.

15. The method of claim 1, wherein the cells associated with the disease are cardiac cells, and wherein the disease is Brugada syndrome, long Q T-syndrome, short Q T-syndrome, atrial standstill, drug induced long Q T, atrial fibrillation, or sick sinus syndrome.

16. The method of claim 1, wherein the set of compounds comprises one or more of disopyramide, ivabradine, veratridine, BAY K 8644, quinidine, amiodarione, propafenone, mexiletine, ajmaline, phenytoin, phenobarbital, carbamazepine, oxcarbazepine, gabapentin, pregabalin, lacosamide, vigabatrin, valproic acid, lamotrigine, topiramate, zonisamide, levetiraacetam, clonazepam, rufinamide, fulnarizine, dalfampridine, acetoazolamide, prednisolone, azathioprine, methotrexate, donepezil, rivastigmine, galantamine, memantine, levodopa, carbidopa, istradefylline, safinamide, pramipexole, rotigotine, ropinirole, amantadine, benztropine, trihexyphyenidyl, selegiline, rasagiline, entacapone, tolcapone, opicapone, choroprozamine, fluphenazine, haloperidol, perphenazine, thioridazine, thiothixene, trifluoperazine, aripiprazole, aripiprazole lauroxil, asenapine, brexpiprazole, cariprazine, clozapine, iloperidone, lumateperonee, lurasidone, olanzapine, samidorphan, paliperidone, paliperidone palmitate, quetiapine, risperidone, ziprasidone, diazepam, hydroxytryptophan, piracetam, nifedipine, flecainide, or sodium valproate.

17. A computer product comprising a non-transitory computer readable medium storing a plurality of instructions that when executed control a computer system to perform:
for each compound of a set of compounds, determining an effect of the compound on a current density of defined ion channels across a cell membrane;
identifying a normal action potential corresponding to a normal state of a cell;
identifying an abnormal action potential corresponding to an abnormal state of the cell;
determining a model that outputs an action potential for an input of current densities of a set of ion channels across the cell membrane;
identifying a first combination of two or more of the set of compounds;
for each compound in the first combination:
determining modified current densities for specified ion channels for a given concentration of the compound, resulting from applying the compound to the abnormal state of the cell;
determining, by the model, a treated action potential using the modified current densities;
determining a difference score between the treated action potential and the normal action potential;
varying the concentration of each compound in the first combination to determine an optimum concentration for each compound in the first combination that results in an optimum difference score;
comparing the optimum difference to a threshold value; and
determining a classification of whether the first combination is a treatment for the disease based on the comparison.

18. The computer product of claim 17, wherein the concentration of each compound is constrained to not exceed a respective concentration threshold when varying the concentration of each compound.

19. The computer product of claim 17, wherein determining the effect of the compound includes retrieving data from a compound database.

20. The computer product of claim 19, wherein determining modified current densities includes inputting the compound concentration to an IC₅₀/EC₅₀ model.
